(19) United States
(12) Patent Application Publication
(10) Pub. No.: US 2018/0028458 A1
(43) Pub. Date: Feb. 1, 2018

(54) COATED CHITOSAN-BASED POLYPLEX FOR DELIVERY OF NUCLEIC ACIDS

(71) Applicant: POLYVALOR, SOCIÉTÉ EN COMMANDITE (S.E.C.), Montreal (CA)

(72) Inventors: Michael D. BUSCHMANN, Montreal (CA); Marc LAVERTU, Pointe-Claire (CA); Monica NELEA, Montreal (CA); Vincent DARRAS, Montreal (CA); Mohamad Gabriel ALAMEH, Montreal (CA); Anik CHEVRIER, Pointe-Claire (CA); Nicolas TRAN-KHANH, Montreal (CA); Ashkan NAEINI TAVAKOLI, Verdun (CA); Daniel VEILLEUX, Montreal (CA)

(73) Assignee: POLYVALOR, SOCIÉTÉ EN COMMANDITE (S.E.C.), Montreal, QC (CA)

(21) Appl. No.: 15/549,971

(22) PCT Filed: Feb. 9, 2016
(86) PCT No.: PCT/CA2016/050119

§ 371 Date: Aug. 9, 2017

Related U.S. Application Data
(60) Provisional application No. 62/113,897, filed on Feb. 9, 2015.
(60) Provisional application No. 62/236,491, filed on Oct. 2, 2015.

(51) Int. Cl. A61K 9/51; A61K 31/713; C12N 15/113

(52) U.S. Cl. CPC A61K 9/5161; A61K 9/5192; C12N 15/113; A61K 31/713; C12N 2310/113; C12N 2320/32; C12N 2310/14; C12N 2310/321

(57) ABSTRACT

The present disclosure relates to a nucleic acid delivery composition comprising a coated chitosan-based polyplex, wherein the coated chitosan-based polyplex comprises: a chitosan; an isolated nucleic acid; and an additional polyelectrolyte. The coated chitosan-based polyplex has an initial or a final molar ratio of amine groups of chitosan (N) to phosphate groups of the nucleic acid (P) to carboxyl groups of the additional polyelectrolyte (C) (N:P:C), wherein the N has a value between about 1.0 and about 10.5, the P has a value between about 1.0 and about 2.0 and the C has a value between about 1.0 and 10.5.

### CROSS-REFERENCE TO RELATED APPLICATIONS

This application claims the benefit of and priority to U.S. provisional patent application No. 62/113,897; filed on Feb. 9, 2015, the content of which is herein incorporated in its entirety by reference; and to U.S. provisional patent application No. 62/236,491, filed on Oct. 2, 2015, the content of which is herein incorporated in its entirety by reference.

### I. FIELD OF TECHNOLOGY

The present disclosure relates to the field of chitosan-based polyplexes for delivery of nucleic acids to cells, tissues or organs. The present disclosure also relates to chitosan-based polyplexes for delivery of nucleic acids to humans and/or animals.

### II. BACKGROUND

Chitosan (CS) is a linear and cationic polysaccharide composed of glucosamine and N-acetyl glucosamine, and is derived from chitin by deacetylation. This cationic polysaccharide holds great interest due to its biocompatibility, biodegradability and mucoadhesive properties (Rinaudo 2006). Chitosan and its derivatives have been proposed for gene delivery applications as they can electrostatically bind to nucleic acid and form nanosized polyelectrolyte complexes which are referred to as polyplexes. The molar mass of the polymer as well as its fraction of ionizable units (its fraction of glucosamine units or its degree of deacetylation) influence its ability to bind nucleic acid and its transfection efficiency (Koping-Hoggard, Varum et al. 2004, Lavertu, et al. 2006).

In order to obtain homogeneous and nanosized structures, CS-based polyplexes are typically prepared by rapidly mixing dilute polycation and nucleic acid solutions (Xu and Anchordoquy 2011). The polycation is generally added in significant excess with respect to the nucleic acid so that the polyplexes produced bear a net positive charge that electrostatically stabilizes them. This positive charge is commonly recognized as desirable for in vitro transfection as it favors non-specific electrostatic interactions with negatively charged cellular membrane. However, a significant fraction of the excess polycation remains unbound and free in the polyplex preparations (Boeckle, von Gersdorff et al. 2004, Ma, Buschmann et al. 2010).

Positively charged polyplexes have shown some efficacy in vivo, (Boeckle, von Gersdorff et al. 2004, Urban-Klein, Werth et al. 2005, Howard, Rahbek et al. 2006, Howard, Paludan et al. 2009, Jean, Alameh et al. 2011), showing only limited colloidal stability at physiological pH and ionic strength and significantly interacting with anionic biomolecules that predominates in biological environment/blood, a potential source of toxicity of this type of delivery system. Some studies suggest that the free polycation present in polyplex formulations of polyethyleneimine (PEI, the most extensively used polycation for nucleic acid delivery) constitutes the principal source of toxicity observed following their intravenous administration at elevated dose (Boeckle, von Gersdorff et al. 2004, Fahrmeir, Gunther et al. 2007). Although the positively charged polyplexes might be better tolerated than the free polycation, they are nevertheless prone to interact with serum proteins and to be rapidly recognized and eliminated by the mononuclear phagocyte system (MPS).

An approach to limit interaction with biomacromolecules and cells found in biological environment is to cover the nanoparticle surface with a polyethylene glycol (PEG) corona (PEGylation) that reduces particle effective charge or zeta potential. PEG is a highly hydrophilic polymer and a sufficiently dense corona will sterically stabilize the nanoparticles and improve their circulation time upon intravenous injection by conferring to them a “stealth” character (slow clearance by MPS) (Jokerst, Lobovkina et al. 2011). PEGylation of polyplex is most often achieved by covalent grafting of PEG to a fraction of polycation's amino groups, but it can also be achieved by synthesizing polycation-β-PEG block copolymers and by covalently reacting PEG chains with available amino groups on the surface of pre-formed polyplexes.

Another approach to limit polyplexes' interactions with biomacromolecules/cells consists of including a hydrophilic polyanion within the polyplex formulation, such that the particles bear a net negative charge.

As such, there remains a need in the art for a delivery system that has limited interactions with biomolecules/cells while maintaining desirable interactions with nucleic acids and that is efficient as a targeted gene/drug delivery carrier.

### III. SUMMARY OF DISCLOSURE

According to various aspects, the present disclosure relates to a nucleic acid delivery composition comprising: a coated chitosan-based polyplex, wherein the coated chitosan-based polyplex comprises: a chitosan; an isolated nucleic acid; and an additional polyelectrolyte; the coated chitosan-based polyplex having an initial or a final molar ratio of amine groups of chitosan (N) to phosphate groups of the nucleic acid (P) to carboxyl groups of the additional polyelectrolyte (C) (N:P:C), wherein the N has a value between about 1.0 and about 10.5, the P has a value between about 1.0 and about 2.0 and the C has a value between about 1.0 and 10.5.

According to various aspects, the present disclosure relates to a method for delivering a nucleic acid to a target comprising the step of contacting the nucleic acid delivery composition as defined herein with the target.

According to various aspects, the present disclosure relates to a method for delivering a nucleic acid to a target in a subject, the method comprising the steps of administering the nucleic acid delivery composition as defined herein to the subject.

According to various aspects, the present disclosure relates to a process for obtaining the nucleic acid delivery composition as defined herein, wherein the process comprises the steps of: a) obtaining a nucleic acid solution; b) obtaining a chitosan solution; c) obtaining an additional polyelectrolyte solution; d) mixing together the solutions of a) and b); e) homogenizing the solution of d); f) adding the solution of c) in the homogenized solution of e); and g) homogenizing the solution obtained in f).

According to various aspects, the present disclosure relates to the use of the nucleic acid delivery composition of any one of claims 1 to 14 for delivering a nucleic acid to a target.

According to various aspects, the present disclosure relates to the use of the nucleic acid delivery composition of any one of claims 1 to 14 for delivering a nucleic acid to a target in a subject.

According to various aspects, the present disclosure relates to the use of a nucleic acid delivery composition for delivering a nucleic acid molecule to the kidney of a subject, wherein the nucleic acid delivery composition comprising a coated chitosan-based polyplex, wherein the coated chitosan-based polyplex comprises: a chitosan; an isolated nucleic acid; and an additional polyelectrolyte; the coated chitosan-based polyplex having an initial or a final molar ratio of amine groups of chitosan (N) to phosphate groups of the nucleic acid (P) to carboxyl groups of the additional polyelectrolyte (C) (N:P:C), wherein the N has a value between about 1.0 and about 10.5, the P has a value

### IV. BRIEF DESCRIPTION OF THE FIGURES

Reference will now be made to the accompanying drawings.

FIG. 1 shows a graph indicating the particle size (Z-Average) and PdI results for HA-coated and uncoated complex suspended in water, and analyzed immediately, and suspended in buffer and analyzed 2 hours after resuspension.

FIG. 2 shows images of a high magnification transmission electron microscopy (TEM) of uncoated (a) vs HA-coated (b) complexes.

FIG. 3 shows images of a high magnification transmission electron microscopy (TEM) of uncoated (a) vs coated (b) HA-coated complexes, coating is visible at high magnification, with 2% phosphotungstate (PTA) (b).

FIG. 4 shows a schematic representation of the in-line mixing platform for production of HA coated chitosan (CS)/NA nanoparticles (continuous mixing configuration).

FIG. 5 shows a graph indicating particle size and PDI of the HA coated CS/ODN complexes in-line mixed and manually mixed using a continuous mixing method (MODE1).

FIG. 6 shows a graph indicating particle size and PDI of the HA coated CS/ODN complexes in-line mixed and manually mixed using a discontinuous mixing method (MODE2).

FIG. 7 shows a graph indicating Zeta potential of the HA coated CS/ODN complexes in-line mixed and manually mixed using continuous and discontinuous mixing methods (MODE1 and MODE2).

FIGS. 8A and 8B show graphs indicating Z-average, PDI and zeta potential of HA-coated siRNA/CS complexes prior to and post freeze-drying following reconstitution at 1×, 10× and 20× their initial concentration.

FIGS. 9A to 9H show images and graphs indicating erythrocyte hemolysis induced by freeze-dried formulations containing CS Mn 10 kDa and 92% DDA without HA and without ODN (A and B), without HA and with ODN (C and D), with HA and without ODN (E and F), with HA and with ODN (G and H).

FIGS. 10A to 10H show images and graphs of erythrocyte hemagglutination induced by freeze-dried formulations containing CS Mn 10 kDa and 92% DDA without HA and without ODN (A and B), without HA and with ODN (C and D), with HA and without ODN (E and F), with HA and with ODN (G and H).

FIG. 11 shows NIRF pictures of a mouse installed in the near-infrared fluorescence (NIRF) imaging system.

FIG. 12 shows images of a ventral view of the in vivo NIRF images acquired at different time points, for HA/CS92-40/siRNA-DY677 NPs. B1: bladder, GB: gall bladder, Int: intestines, Liv: liver.

FIG. 13 shows images of a dorsal view of the in vivo NIRF images acquired at different time points, for HA/CS92-40/siRNA-DY677 NPs. Kid: kidneys, Int: intestines.

FIG. 14 shows ex vivo NIRF images of the liver (top) and the kidneys (bottom), for HA/CS92-10/siRNA-DY677 NPs (left) and for lipid-based NPs (right). GB: gall bladder.

FIG. 15 shows NIRF images of HA/CS92-10 nanoparticles (NPs, arrowheads) localized in renal tubules, 4 h post-injection. The image is from a confocal microscope acquisition of a kidney paraffin section, with the DY677-siRNA fluorescent signal superimposed on tissue autofluorescence.

FIG. 16 shows NIRF images of intracellular localization of HA/CS92-10 nanoparticles (NPs, arrowheads) in epithelial cells of a proximal tubule, 4 h post-injection. The images are from a confocal microscope acquisition of a kidney cryosection. A) The actin stain shows the brush border on the apical side of PTECs and the cell membrane on the basal side, close to the basement membrane. B) Composite image showing NPs and the actin staining. C) Composite image showing the nuclei and the actin staining D) Composite image showing intracellular accumulation of the NPs inside the PTECs. Stains: DY677-siRNA for NPs, AF488-Phalloidin for actin, DAPI for nuclei.

FIG. 17 shows NIRF images of the efficiency of low molecular weight chitosan to effectively deliver EGFP mRNA into the HEK293 cells. The high molecular weight demonstrates very low transfection efficiency when compared to low molecular weight chitosan.

FIGS. 18A and 18B are graphs indicating a physicochemical characterization of chitosan-mRNA nanoparticles. FIG. 18A shows the size and shape of the CS92-10-5 IVT mRNA nanoparticles as measured by DLS and imaged by TEM. The nanoparticles were prepared in water. The average size is around 90 nm and the shape is spherical. 18B shows the encapsulation efficiency and stability of the CS 92-10-5 IVT mRNA versus CS 92-10-5 siRNA nanoparticles in presence of competing polyanions. The chitosan-mRNA nanoparticles are more stable than their siRNA counterparts in presence of high concentration of heparin.

FIG. 19A is a graph showing the particle size and PDI of the HA coated CS/ODN complexes in-line mixed at large scale and manually mixed using a discontinuous mixing method (MODE2).

FIG. 19B is a graph showing Zeta potential of the HA coated CS/ODN complexes in-line mixed at large scale and manually mixed using discontinuous mixing method (MODE2).

FIG. 20 is a graph showing the particle size and PDI of freshly prepared and freeze-dried/concentrated (1× & 20×) HA coated CS/ODN complexes prepared at neutral pH.

FIG. 21 is a graph showing ALT level 24 h following IV administration of siRNA/CS complexes, lipid nanoparticles (Invivofectamine) and controls in CD1 mice.

FIG. 22 is a graph showing AST level 24 h following IV administration of siRNA/CS complexes, lipid nanoparticles (Invivofectamine) and controls in CD1 mice.

FIG. 23 is a graph showing ALP level 24 h following IV administration of siRNA/CS complexes, lipid nanoparticles (Invivofectamine) and controls in CD1 mice.

FIG. 24 is a graph showing BUN level 24 h following IV administration of siRNA/CS complexes, lipid nanoparticles (Invivofectamine) and controls in CD1 mice.

FIG. 25 is a graph showing creatinine level 24 h following IV administration of siRNA/CS complexes, lipid nanoparticles (Invivofectamine) and controls in CD1 mice.

FIG. 26 is a graph showing creatinine kinase level 24 h following IV administration of siRNA/CS complexes, lipid nanoparticles (Invivofectamine) and controls in CD1 mice.

FIG. 27 shows images of an example of absence of histopathological changes in both liver and kidney 24 h following IV administration of siRNA/CS complexes in CD1 mice.

FIG. 28 shows a graph indicating the percentage of lymphocytes measured 24 hours post injection of chitosan-siRNA complexes, LNPs and controls.

FIG. 29 shows a graph indicating the percentage of neutrophils measured 24 hours post injection of chitosan-siRNA complexes, LNPs and controls.

FIG. 30 shows a graph indicating the percentage of basophils measured 24 hours post injection of chitosan-siRNA complexes, LNPs and controls.

In the figures, non-limiting embodiments are illustrated by way of example. It is to be expressly understood that the description and drawings are only for the purpose of illustrating certain embodiments and are an aid for understanding. The scope of the claims should not be limited by the embodiments set forth in the present disclosure, but should be given the broadest interpretation consistent with the description as a whole.

### V. DETAILED DESCRIPTION

In accordance with the present disclosure, there is provided a system for delivery of nucleic acids to a target. In some embodiments, the target is a population of cells, a tissue, or an organ. In some embodiments, the target is a human or an animal.

In some implementations of these embodiments, the delivery system is a chitosan-based polyplex. In some instances, the chitosan-based polyplex is a coated chitosan-based polyplex.

By “chitosan-based polyplex” or “polyplex” is meant a complex comprising a plurality of chitosan molecules (each a polymer of glucosamine monomers) and a plurality of nucleic acid molecules.

The delivery system of the present disclosure may be used for a variety of purposes, such as for example, but not limited to, studying the function of a target transcript, studying the effect of different compounds of a cell or organism in the absence of, or with reduced activity of, the polypeptide encoded by the transcript.

In some instances, the delivery system of the present disclosure may be useful for down-regulating the expression of molecules that are overexpressed in, for example, cancer, renal disease, liver diseases, cardiovascular disease, genetic diseases, viral infection, neuromuscular disease, neurodegenerative disease, inflammatory disease, arthritis, metabolic disease, or diabetes.

Furthermore, the delivery system of the present disclosure may be useful in clinical therapy for various diseases and conditions such as, but not limited to, cancer, renal disease, liver diseases, cardiovascular disease, genetic diseases, viral infection, neuromuscular disease, neurodegenerative disease, inflammatory disease, arthritis, metabolic disease, diabetes.

The present disclosure also relates to methods of preventing and/or treating diseases or conditions associated with excessive expression or with inappropriate expression of a target transcript; or inappropriate or excessive activity of a polypeptide encoded by the target transcript or to correct genetic mutations by delivering to a target the chitosan-based polyplex of the present disclosure.

The delivery system of the present disclosure may be used, for example, to provide symptomatic relief, by administering a nucleic acid using the delivery system disclosed herein to a subject at risk of, or, suffering from such a condition within an appropriate time window prior to, during, or after the onset of symptoms.

In some specific implementations, the chitosan-based polyplex of the present disclosure may be useful in the prevention and/or treatment of lymphomas.

In some specific implementations, the chitosan-based polyplex of the present disclosure may be useful to deliver nucleic acids to the kidney.

In some specific implementations, the chitosan-based polyplex of the present disclosure may be useful in the prevention and/or treatment of fibrosis.

In some specific implementations, the chitosan-based polyplex of the present disclosure may be useful to deliver nucleic acids to the liver.

In some specific implementations, the chitosan-based polyplex of the present disclosure may be useful in the prevention and/or treatment of liver-related diseases such as, but not limited to, liver cancer. In some implementations, the liver cancer could be hepatocellular carcinoma (HCC). Animal models may be used in order to study tumorigenesis and/or the assessment of molecules for the development of therapies against HCC. The most used models include xenograft and orthotopic models of cancer. Anti-miR21 has been proposed for HCC treatment.

In some specific implementations, the chitosan-based polyplexes of the present disclosure may be useful in the prevention and/or for treatment of kidney-related diseases such as, but not limited to, renal fibrosis. Renal fibrosis is the final common pathway for most forms of progressive renal disease, and involves glomerular sclerosis and/or interstitial fibrosis. Animal models used for testing of acute and chronic kidney diseases include the unilateral ureteral obstruction (UUO) model, the ischemic-reperfusion model and the acute/chronic fibrosis models including models of immune or nephrotoxin induced fibrosis.

In general, regulatory pathways and pathophysiological changes associated with human diseases have been studied by the use of transgenic mice models used as a background for the generation of fibrosis. The use of such models permits the effect of genes/pathways in the development of disease to be studied. New therapeutic molecules such as siRNA or anti-miR (such as for example, anti-miR192 and anti-miR-29b) have been used to study the pathophysiology in the above said models. Modified mRNA, pDNA or minicircle pDNA may also be used to express therapeutic proteins rather than siRNA for inhibition.

As used herein, the terms “treatment” and “treating” include preventing, inhibiting, and alleviating symptoms of a disease, disorder or condition. The treatment may be carried out by administering a therapeutically effective amount of the composition described herein. Moreover the delivery system as described herein can be used in conjunction with any other treatment such as for example any other cancer treatment (e.g., radiotherapy, surgery, hormonal treatment or conventional chemotherapy).

In some implementations, the chitosan-based polyplex is a chitosan-based nanoparticle. Depending on conditions under which they are produced, the nanoparticles have an average diameter of between about 10 nm and about 500 nm.

In some implementations, the chitosan-based polyplex comprises a chitosan residue and a nucleic acid molecule. In some instances, the chitosan-based polyplex comprises a chitosan residue, a nucleic acid molecule and one or more additional polyelectrolyte(s).

As used herein, the term “polyelectrolyte” refers to a polymer whose repeating units bear an electrolyte group. The polyelectrolyte of the present disclosure may be a polyanion. In some instances, the additional polyelectrolyte is not a chitosan or a nucleic acid molecule. In some instances, the additional polyelectrolyte is a compound that coats the polyplex. As used herein, the expression “coated chitosan-based polyplex” refers to a polyplex that comprises chitosan, a nucleic acid and an additional polylectrolyte, polyanion or polyampholyte.

As used herein, the expression “chitosan residue” or “CS” generally refers to a chitosan residue having a deacetylation degree (% DDA) from about 50% to about 100% and/or a molecular weight (Mn) of from about 2 kDa to about 200 kDa.

The expression “chitosan residue” may also generally refer to any modified chitosan residue where the modification(s) is either on the chitosan lateral amines and/or on the chitosan hydroxyl groups.

The person of skill will readily envision the types of modifications which can be suitable for this purpose.

The molecular weight and the degree of deacetylation (DDA) of chitosan indicate its biological and physicochemical properties. The degree of deactylation of chitosan is the percentage of glucosamine monomers (100% DDA is polyglucosamine while 80% DDA has 80% glucosamine and 20% N-acetyl-glucosamine). For example, chitosan biodegradability is affected by the amount and the distribution of acetyl groups. The absence of these groups or their random, rather than block, distribution results in a lower rate of degradation. Prior studies have addressed the effect of chitosan molecular weight (Mn) and degree of deacetylation (DDA) on nanoparticle uptake, nanoparticle trafficking, and transfection efficiency on different cell lines. A study addressing this complex relationship has been achieved by Lavertu et al. (Biomaterials, 27: 4815-4824). In their study, they varied the molecular weight and the DDA systematically and independently as well as the molar ratio of amine groups of the chitosan (N) to the phosphate groups of the nucleic acid (P) (ratio N:P) and/or the pH of the transfection media.

The combined effect of the chitosan formulation parameters (DDA, Mn, N:P), also referred to herein as the “DDA-Mn-N:P signature” was studied by Lavertu et al. (2006, Biomaterials, 27: 4815-4824). They found that maximum transgene expression occurs for DDA:Mn values that run along a diagonal from high DDA/low Mn to low DDA/high Mn. Thus if one increases/decreases DDA, one must correspondingly decrease/increase Mn to maintain maximal transfection. pH plays an important role in transfection efficiency since an increase in pH displaces the Mn for the most efficient formulation toward higher Mn because of the destabilization effect of pH by reducing chitosan protonation. On the other hand, for a given DDA, a change in N:P ratio from 5:1 to 10:1 displaces the Mn for the most efficient formulation towards lower Mn, probably because of the stabilizing effect of increasing chitosan concentration.

As used herein, “average weight” of chitosan polymers refers to the number average molecular weight. In some instances, the chitosan in the chitosan-based polyplex of the present disclosure has a Mn of between about 2 kDa to about 200 kDa, preferably between about 10 kDa and 150 kDa. In some instances, the chitosan in the chitosan-based polyplex of the present disclosure has a Mn of of about 10 kDa, about 40 kDa, about 80 kDa, about 120 kDa, or about 150 kDa.

In some instances, the chitosan in the chitosan-based polyplex of the present disclosure has a DDA of between about 70% and about 100%, preferably between about 70% and about 99%, preferably between about 72% and about 100%, preferably between about 72% and about 99%, more preferably between about 72% and about 98%. In some instances, the chitosan in the chitosan-based polyplex of the present disclosure has a DDA of about 98%, about 92%, about 80% or about 72%.

In some instances, the chitosan in the chitosan-based polyplex of the present disclosure has an N:P ratio that is about 1.5:1, about 1.5:1.5, about 2:1, about 2:1.5, about 2.5:1, about 2.5:1.5, about 3:1, about 3:1.5, about 3.5:1, about 3.5:1.5, about 4:1, about 4:1.5, about 4.5:1, about 4.5:1.5, about 5:1, about 5:1.5, about 5.5:1, about 5.5:1.5, about 6:1, about 6:1.5, about 6.5:1, about 6.5:1.5, about 7:1, about 7:1.5, about 7.5:1, about 7.5:1.5, about 8:1, about 8:1.5, about 8.5:1, about 8.5:1.5, about 9:1, about 9:1.5, about 9.5:1 about 9.5:1.5 about 10:1 or about 10:1.5.

In some implementations, the chitosan of the polyplex of the present disclosure has a DDA-Mn-N:P signature selected from the signatures identified Table 1:

TABLE 1
Examples of DDA-Mn-N:P signatures of chitosan

| Mn | DDA | N:P |
|---|---|---|
| 10 | 98 | 2:1 |
| 10 | 92 | 2:1 |
| 10 | 80 | 2:1 |
| 10 | 72 | 2:1 |
| 40 | 98 | 2:1 |
| 40 | 92 | 2:1 |
| 40 | 80 | 2:1 |
| 80 | 98 | 2:1 |
| 80 | 92 | 2:1 |
| 80 | 80 | 2:1 |
| 80 | 72 | 2:1 |
| 120 | 98 | 2:1 |
| 120 | 92 | 2:1 |
| 120 | 80 | 2:1 |
| 120 | 72 | 2:1 |
| 150 | 98 | 2:1 |
| 150 | 92 | 2:1 |
| 150 | 80 | 2:1 |
| 150 | 72 | 2:1 |
| 10 | 98 | 5:1 |
| 10 | 92 | 5:1 |
| 10 | 80 | 5:1 |
| 10 | 72 | 5:1 |
| 40 | 98 | 5:1 |
| 40 | 92 | 5:1 |
| 40 | 80 | 5:1 |
| 40 | 72 | 5:1 |
| 80 | 98 | 5:1 |
| 80 | 92 | 5:1 |
| 80 | 80 | 5:1 |
| 80 | 72 | 5:1 |
| 120 | 98 | 5:1 |
| 120 | 92 | 5:1 |
| 120 | 80 | 5:1 |
| 120 | 72 | 5:1 |
| 150 | 98 | 5:1 |
| 150 | 92 | 5:1 |
| 150 | 80 | 5:1 |
| 150 | 72 | 5:1 |
| 10 | 98 | 10:1 |
| 10 | 92 | 10:1 |
| 10 | 80 | 10:1 |
| 10 | 72 | 10:1 |
| 40 | 98 | 10:1 |
| 40 | 92 | 10:1 |
| 40 | 80 | 10:1 |
| 40 | 72 | 10:1 |
| 80 | 98 | 10:1 |
| 80 | 92 | 10:1 |
| 80 | 80 | 10:1 |
| 80 | 72 | 10:1 |
| 120 | 98 | 10:1 |
| 120 | 92 | 10:1 |
| 120 | 80 | 10:1 |
| 120 | 72 | 10:1 |
| 150 | 98 | 10:1 |
| 150 | 92 | 10:1 |
| 150 | 80 | 10:1 |
| 150 | 72 | 10:1 |

The nucleic acid of the chitosan-based polyplex of the present disclosure may be a deoxyribonucleic acid (DNA) or may be a ribonucleic acid (RNA). Such DNA or RNA may be single- or double-stranded. For example, the nucleic acid may be a plasmid DNA, a vector DNA, a minicircle DNA, a messenger RNA (mRNA), a modified mRNA, siRNA, modified siRNA or a microRNA (miRNA). The nucleic acid may be isolated from cells, may be made by synthetic methods known in the art or may be transcribed in vitro.

In some instance, the nucleic acid of the present disclosure may be modified. For example, the nucleic acid may be modified on its backbone. Examples of modifications that can be performed on the backbone of a nucleic acid include, but are not limited to, phosphorothioate (PS), boranophosphate, phosphonoacatate (PACE), morpholine, peptide nucleic acid backbone modification (PNA), and amid-linked bases. The nucleic acid may also be modified on the sugar moiety and/or on the base moiety. Examples of modifications that can be performed on the sugar and/or the base moieties include, but are not limited to, locked nucleic acid (LNA), phosphoramidate (NP), 2′F-RNA, 2′-0 methoxyethyl (2′MOE), 2′O-methyl (2′OMe), 2′-O-fluoro (2′-F) 5-bromouracil, 5-iodouracil, 5-methylcytosine, ethylene bridged nucleic acids (ENA), diaminopurine, 2-thiouracil, 4-thiouracil, pseudouracil, hypoxantine, 2-aminoadenine, 6-methyl or other alkyl derivates of adenine and guanine, 2-propyl and other derivative of adenine and guanine, 6-azo-uracil, 8-halo, 8-amino, 8-thiol, 8-hydroxyk and other 8-substituted adenines and guanines, constrained ethyl sugar moiety (cET), ribofuranosyl, 2′-0,4′-C-methylene and 2′-0,4′-C-ethylene bicyclic nucleotide analogs, acyclic nucleotides (UNA and PNA), and dihydrouridine modification. Other modifications that may be performed on nucleic acids are, but are not limited to, modifications that include deoxyribonucleotide bases incorporated in a ribonucleotide sequence. The incorporations may be limited to the overhang structure in the canonical siRNA architecture or may be distributed in the sequence.

Modifications to RNA molecules include, but are not limited to blunt-ended siRNA, 25-27mer siRNA, single strand siRNA, short hairpin siRNA, dumbbell siRNA, asymetric siRNA, short interspaced siRNA, hybrid between siRNA and antisense oligonucleotides (ASO).

Other analog nucleic acids may be contemplated include those with non-ribose backbones. In addition, mixtures of naturally occurring nucleic acids, analogs, and both may be made. Nucleic acids include but are not limited to DNA, RNA and hybrids where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 5-methylcytidine, pseudouridine etc. Modified 5′ cap structures such as 3′-O-Me-m7G(5′)ppp(5′)G (anti-reverse cap analog), may also be used for increased translation of mRNA. Nucleic acids include DNA in any form, RNA in any form, including triplex, duplex or single-stranded, antisense, siRNA, ribozymes, deoxyribozymes, polynucleotides, oligonucleotides, chimeras, and derivatives thereof.

In some implementations of the nucleic acid is a plasmid or a vector DNA. In some instances, the plasmid or vector DNA comprises a sequence that encodes for a therapeutic molecule such a therapeutic protein. For example, the plasmid or vector DNA may comprise a sequence that encodes for a component involved in gene editing such as a component of the clustered regularly interspaced short palindromic repeats (CRISPRs)/Cas gene system.

In one embodiment, the nucleic acid component may comprise a therapeutic nucleic acid. Therapeutic nucleic acids include therapeutic RNAs, which are RNA molecules capable of exerting a therapeutic effect in a mammalian cell. Therapeutic RNAs include antisense RNAs, siRNAs, short hairpin RNAs, and enzymatic RNAs. Therapeutic nucleic acids include nucleic acids intended to form triplex molecules, protein binding nucleic acids, ribozymes, deoxyribozymes, and small nucleotide molecules. Therapeutic nucleic acids also include nucleic acids encoding therapeutic proteins, including cytotoxic proteins and prodrugs; ribozymes; antisense or the complement thereof; or other such molecules.

The nucleic acid component may comprise a therapeutic nucleic acid construct. The therapeutic nucleic acid construct is a nucleic acid construct capable of exerting a therapeutic effect. Therapeutic nucleic acid constructs may comprise nucleic acids encoding therapeutic proteins, as well as nucleic acids that produce transcripts that are therapeutic RNAs. A therapeutic RNA is an RNA molecule capable of exerting a therapeutic effect in a mammalian cell. Therapeutic RNAs include antisense RNAs, siRNAs, short hairpin RNAs, enzymatic RNAs, and messenger RNAs. Therapeutic nucleic acids include nucleic acids intended to form triplex molecules, protein binding nucleic acids, ribozymes, deoxyribozymes, and small nucleotide molecules. A therapeutic nucleic acid may be used to effect genetic therapy by serving as a replacement or enhancement for a defective gene or to compensate for lack of a particular gene product, by encoding a therapeutic product. A therapeutic nucleic acid may also inhibit expression of an endogenous gene. A therapeutic nucleic acid may also encode all or a portion of a translation product, and may function by recombining with DNA already present in a cell, thereby replacing a defective portion of a gene. It may also encode a portion of a protein and exert its effect by virtue of co-suppression of a gene product.

In some instances, the nucleic acids are RNAs that perform a biological function when introduced into cells such as messenger RNAs and self-replicating mRNAs, also referred to as replicon RNA. Also preferred are ribonucleic acids that have biological effects when introduced into cells such as antisense RNAs or interfering RNA, including long double-stranded RNA and small interfering RNA (siRNA), that can inhibit the function of an RNA endogenous to a cell containing a sequence that can hybridize or otherwise form a complex with the interfering RNA or antisense RNA.

In some implementations, the nucleic acid is a microRNA (miRNA or miRs). miRNA's are typically 8-30 bases long oligonucleotide that regulate protein expression through binding to the 3′untranslated region (3′UTR) of messenger RNA (mRNA). To date, more than 1000 miRNAs have been identified and most of them had their role elucidated in normal and pathological pathways. The importance of miRNA biology is highlighted by findings that their expression can be modulated/regulated in pathology and by their ability to bind many target mRNAs. Through multiple target binding, a single miRNA can regulate a whole/specific transcriptomic program; i.e., over expression of some target and repression of other. miRNA overexpression has been demonstrated in many fibrotic diseases and cancer. For instance, overexpression of miRNA 21 has been shown to increase collagen production and deposition in fibrosis. In cancer, specifically in hepatocellular carcinoma, miRNA21 has been demonstrated to be upregulated and consequently its repression promotes tumor reduction via downregulation of tumor promoting genes.

In some implementations, the additional polyelectrolyte comprised in the chitosan-based polyplex is a polyanion. The polyanion may be any anion containing a plurality of negative charges at the pH value at which particle formation occurs. Specific examples of useful polyanions include the sulfate anion, oligophosphates such as tripolyphosphate (TPP), nucleoside triphosphate including adenosine triphosphate (ATP), nucleoside diphosphates including adenosine diphosphate (ADP), poly-acrylic acid, chondroitin sulfate, keratan sulfate, dermatan sulfate, alginate, hyaluronate, dextran sulfate, heparin, heparan sulfate, gellan gum, pectin, kappa, lamda and iota carrageenan, xanthan and derivatives thereof; sulfated, carboxymethylated, carboxyethylated or sulfoethylated varieties of glucans or xylans, glucan or xylan derivatives, glycosaminoglycans or glycosaminoglycan derivatives; and polyampholytic proteins like collagen and keratose.

In some instances, the N:P portion of the DDA-Mn-N:P signature of the polyplex of the present disclosure is expressed as N:P:C, wherein N is moles of amine (N) of chitosan, P is moles of phosphates (P) of the nucleic acid, and C is moles of carboxyl (C) of the additional polyanion.

The N:P:C ratio of the polyplex of the present disclosure reflects the N of chitosan, the P of the nucleic acid and the C of the additional polyanion prior to combining the chitosan, nucleic acid and/or the additional polyelectrolyte (N:P:C initial). It is noted that the N:P:C does not represent the final composition when removal of excess components by diafiltration or else is required.

In some instances, the chitosan in the chitosan-based polyplex of the present disclosure has an N:P:C ratio that is about 1:1:0.25, about 1:1:0.5, about 1:1:0.75, about 1:1:1, about 1:1:1.5, about 1:1:1.75, about 1.5:1:0.25, about 1.5:1:0.5, about 1.5:1:0.75, about 1.5:1:1, about 1.5:1:1.5, about 1.5:1:1.75, about 2:1:0.25, about 2:1:0.5, about 2:1:0.75, about 2:1:1, about 2:1:1.5, about 2:1:1.75, about 2.5:1:0.75, about 2.5:1:1, about 2.5:1:1.5, about 2.5:1:1.75, about 3:1:0.75, about 3:1:1, about 3:1:1.5, about 3:1:1.75, about 3.5:1:0.75, about 3.5:1:1, about 3.5:1:1.5, about 3.5:1:1.75, about 4:1:0.75, about 4:1:1, about 4:1:1.5, about 4:1:1.75, about 4.5:1:0.75, about 4.5:1:1, about 4.5:1:1.5, about 4.5:1:1.75, about 5:1:0.75, about 5:1:1, about 5:1:1.5, about 5:1:1.75, about 5.5:1:0.75, about 5.5:1:1, about 5.5:1:1.5, about 5.5:1:1.75, about 6:1:0.75, about 6:1:1, about 6:1:1.5, about 6:1:1.75, about 6.5:1:0.75, about 6.5:1:1, about 6.5:1:1.5, about 6.5:1:1.75, about 7:1:0.75, about 7:1:1, about 7:1:1.5, about 7:1:1.75, about 7.5:1:0.75, about 7.5:1:1, about 7.5:1:1.5, about 7.5:1:1.75, about 8:1:0.75, about 8:1:1, about 8:1:1.5, about 8:1:1.75, about 8.5:1:0.75, about 8.5:1:1, about 8.5:1:1.5, about 8.5:1:1.75, about 9:1:0.75, about 9:1:1, about 9:1:1.5, about 9:1:1.75, about 9.5:1:0.75, about 9.5:1:1, about 9.5:1:1.5, about 9.5:1:1.75, about 10:1:0.75, about 10:1:1, about 10:1:1.5, about 10:1:1.75, about 10.5:1:0.75, about 10.5:1:1, about 10.5:1:1.5, or about 10.5:1:1.75.

In some instances, the P:C ratio of the polyplex of the present disclosure reflects the P of the nucleic acid and the C of the additional polyelectrolyte prior to combining the nucleic acid and the additional polyelectrolyte (P:C initial). The P:C initial is used, for example, when diafiltration of excess components is required.

In some instances, the P:C ratio of the polyplexes of the present disclosure reflects the P of the nucleic acid and the C of the additional polyelectrolyte after combining the nucleic acid and the additional polyelectrolyte (P:C final). The P:C initial is used, for example, when diafiltration of excess components is not required.

In some instances, the chitosan in the chitosan-based polyplex of the present disclosure has an P:C ratio that is about 1:1, about 1:1.5, about 1:1.75, about 1.5:1, about 1.5:1.5 or about 1.5:1.75.

In some implementations, the polyelectrolyte is hyaluronic acid (HA). Hyaluronic acid or hyaluronan is a highly hydrophilic natural polyanion composed of repeating disaccharides of N-acetyl glucosamine and glucuronate. Some membrane receptors are known to bind HA (CD44, RHAMM, HARE and LYVE-1) and these receptors are abundant in liver, kidney, spleen, eye and most cancer tissues (Oh, Park et al. 2010). HA may be incorporated into the chitosan-based polyplex preparation using different approaches. HA may be added to the nucleic acid solution (with a multivalent anionic electrostatic cross-linker, namely tripolyphosphate or TPP to create electrostatic complex by the so-called ionotropic gelation method) prior to mixing with CS (de la Fuente, Seijo et al. 2008, Contreras-Ruiz, de la Fuente et al. 2011, Gwak, Jung et al. 2012, Al-Qadi, Alatorre-Meda et al. 2013, Oliveira, Bitoque et al. 2014). Although some of the polyplexes prepared using this one step mixing method are reported to be more stable against protein adsorption than the “native” binary polyplexes, HA is likely to be entrapped within such structures and this could limit their targeting efficiency as well as their colloidal stability and their “stealth” character.

Other authors have included HA within their delivery systems by first mixing CS and HA/TPP solutions using an excess of CS. The nucleic acid was incorporated to the resulting positively charged nanoparticles by simply subsequently adding the nucleic acid (Duceppe and Tabrizian 2009, Lu, Zhao et al. 2011, Lu, Lv et al. 2013). As for the one step mixing method described above, HA is likely to be entrapped within the resulting structure and such an approach presents similar drawbacks, in addition to exposing the nucleic acid that coats the complexes to the biological environment.

The order of steps that are performed to produce the polyplex of the present disclosure may vary. In one embodiment, a solution comprising the chitosan and a solution comprising the nucleic acid may be combined as described herein with a solution comprising the additional polyelectrolyte. In some other instances, a solution comprising an additional polyelectrolyte and a nucleic acid may be combined as described here with a solution of a chitosan.

Amounts of components combined are chosen such that polyplexes with the desirable N:P, P:C and/or N:P:C ratios are obtained. Another method is to combine a solution comprising a nucleic acid and, optionally, an additional polyelectrolyte with a solution comprising a chitosan such that nanoparticles of desirable N:P, P:C and/or N:P:C ratios are obtained. Any excess of uncomplexed chitosan may be removed by processes such as, but not limited to, dialysis, ultrafiltration and centrifugation.

It is noted that additional components can be added during polyplex formation. Examples of such additional components, include, but are not limited to, are multivalent cations such as calcium, tripolyphosphate (TPP), uncharged polymers such as polyethylene glycol, or uncharged saccharide derivatives. Additional components may also include one or more biologically active substances. Such biologically active substances may be any biologically active substance, including small-molecule drugs or pro-drugs and therapeutic or otherwise biologically active peptides or proteins, provided that they are soluble in aqueous solutions at concentrations exceeding the concentrations at which they are therapeutically active or exert their other biological activity.

The present disclosure also relates to pharmaceutically acceptable or physiologically acceptable compositions or formulations comprising chitosan-based polyplexes of the present disclosure. Such compositions or formulations may be in a form suitable for administration to a target such as a subject in the context of, for example, a treatment method.

As used herein, the expressions “pharmaceutically acceptable” and “physiologically acceptable” refer to carriers, diluents, excipients and the like that can be administered to a subject, preferably without producing excessive adverse side-effects. Such preparations for administration preferably include sterile aqueous or non-aqueous solutions, suspensions, and emulsions. Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like. A pharmaceutical formulation can be formulated to be compatible with its intended route of administration. For example, for oral administration, a composition can be incorporated with excipients and used in the form of tablets, troches, or capsules, e.g., gelatin capsules. Pharmaceutically compatible binding agents, and/or adjuvant materials can be included in oral formulations. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or flavoring.

Formulations can also include carriers to protect the composition against rapid degradation or elimination from the body, such as a controlled release formulation, including implants and microencapsulated delivery systems. For example, a time delay material such as glyceryl monostearate or glyceryl stearate alone, or in combination with a wax, may be employed.

Any of a number of administration routes are possible and the choice of a particular route will in part depend on the target tissue.

The doses or “effective amount” for treating a subject are preferably sufficient to ameliorate one, several or all of the symptoms of the condition, to a measurable or detectable extent, although preventing or inhibiting a progression or worsening of the disorder or condition, or a symptom, is a satisfactory outcome. Thus, in the case of a condition or disorder treatable by expressing a therapeutic nucleic acid in target tissue, the amount of therapeutic RNA or therapeutic protein produced to ameliorate a condition treatable by a method of the present disclosure will depend on the condition and the desired outcome and can be readily ascertained by the skilled artisan. Appropriate amounts will depend upon the condition treated, the therapeutic effect desired, as well as the individual subject (e.g., the bioavailability within the subject, gender, age, etc.). The effective amount can be ascertained by measuring relevant physiological effects.

The compounds of the present disclosure may be administered orally. Oral administration may involve swallowing, so that the compound enters the gastrointestinal tract. Compositions of the present disclosure may also be administered directly to the gastrointestinal tract

Formulations suitable for oral administration include solid formulations such as tablets, capsules containing particulates, liquids, or powders, lozenges (including liquid-filled), chews, multi- and nano-particulates, gels, films, ovules, and sprays.

Liquid formulations include suspensions, solutions, syrups and elixirs. Liquid formulations may be prepared by the reconstitution of a solid.

Tablet dosage forms generally comprise a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose.

Solid formulations for oral administration may be formulated to be immediate and/or modified release. Modified release formulations include delayed-, sustained-, pulsed-, controlled-, targeted and programmed release.

The compounds of the present disclosure may also be administered directly into the blood stream, into muscle, or into an internal organ. Suitable means for parenteral administration include intravenous, intraarterial, intraperitoneal, intrathecal, intraventricular, intraurethral, intrasternal, intradermal, intra-articular, intracranial, intramuscular and subcutaneous. Suitable devices for parenteral administration include needle (including microneedle) injectors, needle-free injectors and infusion techniques.

The compounds of the present disclosure may also be administered directly to the eye or ear, typically in the form of drops. Administration into the eye may be to the front (the cornea) or to the back (the vitreous and the retina). Other formulations suitable for ocular and aural administration include ointments, biodegradable (e.g. absorbable gel sponges, collagen) and non-biodegradable (e.g. silicone) implants, wafers, lenses and particulate systems. Formulations may also be delivered by iontophoresis.

Parenteral formulations are typically aqueous solutions which may contain excipients such as salts, carbohydrates and buffering agents, but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

In some embodiments, the polyplexes of the present disclosure may be freeze dried. In some implementations of this embodiment, freeze dried polyplexes of the present disclosure may be concentrated upon rehydration without changes in the biological activity or creation of hyperosmotic solutions, provided that appropriate lyoprotectant type and concentration, and buffer type and concentration, are present in the particle suspension to be lyophilized. The techniques for freeze drying and lyophilization of the chitosan-nucleic acid complexes are described in WO 2014/197970, which is incorporated herein by reference.

### VI. EXAMPLES

### Example 1

### Coated Polyplexes

Chitosans with a degree of deacetylation of 92%, and Mn of 10, 40 and 120 kDa were produced by alkaline deacetylation and nitrous acid depolymerization. Sodium hyaluronate powder (HA, Mn=866 kDa) was obtained from Lifecore Biomedical.

siRNA ApoB (also referred to as ApoB native): sense: 5′-GUCAUCACACUGAAUACCAAU-3′, antisense: 5′-AUUGGUAUUCAGUGUGAUGACAC-3′ and siRNA-DY677 (fluorescently labeled): sense: 5′-DY677-AGAUGAGGUGUCCUGCAAACC-3 ‘, antisense: 5’-UUUGCAGGACACCUCAUCUGG-3′ were obtained from Thermo Scientific. ODN ApoB: sense: 5′-GTCATCACACTGAATACCAAT-3′, antisense: 5′-ATTGGTATTCAGTGTGATGACAC-3′, was obtained from Integrated DNA Technologies. HCl 1N (Product No 318949), Trehalose, (Product No 90208), L-histidine (Product No H6034-25G), HEPES free acid (Product No H-4034), hexacyanoferrate (III) (Product No 31253-250G), potassium cyanide (Product No 31252-100G), potassium phosphate monobasic (Product No 04243-500G), Triton X-100 (Product No T-8532) and phosphotungstic acid hydrate (Product No T-79690-25G) were from Sigma. Ultra-pure RNAse and DNAse Free ddH₂O (Product No 10977023), Invivofectamine kit, (Product No 1377505) and Phosphate-Buffered Saline (PBS) pH 7.4 (Product No 10010-031) were from Life Technologies. LPS from E. coli, Serotype 055:B5 (TLRgrade™, Product No ALX-581-013-L002) and polyinosinic-polycytidylic acid potassium salt or Poly(I:C) (synthetic, TLRgrade™, Product No ALX-746-021-M002) were from Enzo life Science. Isoflurane, USP (Product No 1001936060) was from Baxter. TEM grids, Carbon film on 200 Mesh Copper Grids (Product No CF200-Cu) were from EMS.

### Example 2

### Manual Production and Stability Assessment of HA-Coated Polyplexes

Preparation of Nucleic Acid Solution:

siRNA ApoB was obtained sterile and rehydrated using RNase free water to obtain a final concentration of 1 mg/mL. siRNA stock solution was diluted to 200 μg/ml using RNase free water, filtered 4% (w/v) trehalose dihydrate, and filtered 28 mM L-histidine buffer at pH 6.5. Trehalose and L-histidine final concentrations were 0.5% w/v and 3.5 mM, respectively.

Preparation of Chitosan Solution:

Chitosan (Mn 10 kDa, 92% DDA) was dissolved in HCl overnight at room temperature to obtain a final chitosan concentration of 5 mg/mL and filtered using 0.2 μm filter. The chitosan stock solution was diluted to 225 μg/ml with RNase free water, filtered 4% (w/v) trehalose dihydrate, and filtered 28 mM L-histidine buffer at pH 6.5. Trehalose and L-histidine final concentrations were 0.5% w/v and 3.5 mM, respectively.

Preparation of HA Solution:

Sodium hyaluronate with molecular weight (Mn) of 866 kDa was dissolved in RNase free water at room temperature at 1.25 mg/mL. The HA stock solution was diluted to 390 μg/mL using RNase free water, filtered 4% (w/v) trehalose dihydrate, and filtered 28 mM L-histidine buffer at pH 6.5, and sterilized using 0.2 μm filter. Trehalose and L-histidine final concentrations were 0.5% w/v and 3.5 mM, respectively.

Manual Production of Polyplexes: Uncoated CS92-10/siRNA Samples:

Complexes were prepared using a CS having a N:P ratio of 2:1, (N:P is the molar ratio of amines (N) of chitosan, to the phosphates (P) of the nucleic acid) as follows: 200 μL of each of siRNA dilution were pipetted and transferred to a 1.5 mL RNase free centrifuge tube. 200 tit of the chitosan solution were pipetted into the siRNA solution and the mixture was immediately pipetted up and down ˜10× for homogenization. The prepared polyplex solution was incubated for 30 minutes at room temperature. 200 μL of water were added to the polyplex solution. Afterwards, 300 μL were resuspended in water and 300 μL were resuspended in a buffer solution (20 mM HEPES+150 mM NaCl, pH7.4) for DLS analyses.

Manual Production of Polyplexes: HA Coated CS92-10/siRNA Samples:

Complexes were prepared using a CS having a N:P:C ratio of 2:1:1.5 (N:P:C is the molar ratio of amines (N) of chitosan, to the phosphates (P) of the nucleic acid, to the carboxyls (C) of HA) as follows: 200 μL of siRNA dilution were pipetted and transferred to a 1.5 mL RNase free centrifuge tube. 200 μL of the chitosan solution were pipetted into the siRNA solution and the mixture was immediately pipetted up and down ˜10× for homogenization. The polyplex solution was then incubated for 30 minutes at room temperature. Afterwards, 200 μL of the HA solution were added to the CS/siRNA polyplex solution and homogenised by pipetting as above. The prepared mixture was incubated for 30 minutes at room temperature. Afterwards, 300 μL were resuspended in water and 300 μL were resuspended in a buffer solution (20 mM HEPES+150 mM NaCl, pH7.4) for DLS analyses.

Complexes Physicochemical Characterization:

Dynamic Light Scattering (DLS) was used to measure the size and polydispersity of the prepared nanoparticles (polyplexes). 400 μL of each sample were transferred into the size cuvette. The instrument was adjusted for three size measurements on each sample. Mean values of Z-Average, mean intensity-weighted size and PdI (average of the three DLS readings performed for each sample) were calculated. The standard deviation and also the coefficient of variation (CV %) were calculated for all the samples. Transmission Electron Microscopy (TEM) was used to assess size and morphology of the polyplexes and to visualize the HA coating. For TEM analyses, a drop of solution was pipetted on the TEM grid then the excess sample solution was dried by capillarity on a filter paper. Some samples were stained to better visualise the HA coating: a drop of phosphotungstatic acid was pipetted on the grid and incubated for 2 minutes. The excess solution was then removed by capillarity using filter paper.

DLS and TEM Results:

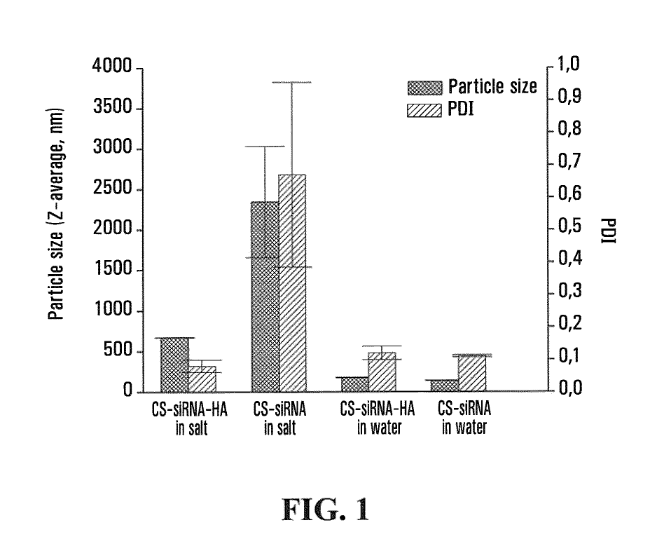

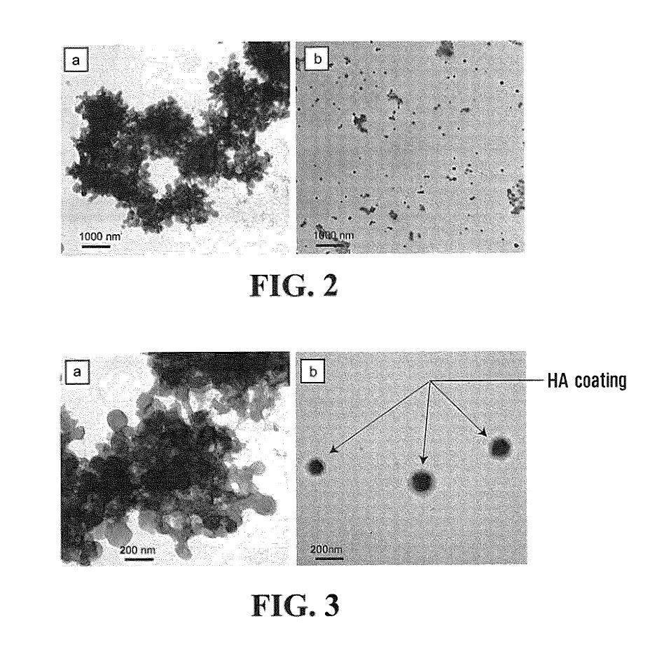

Complexes were analyzed in DLS, after resuspension in water and 2 hours after resuspension in buffer (20 mM HEPES+150 mM NaCl, pH7.4). The complexes incubated 2 hours in buffer were analyzed by TEM. In DLS (size and PdI), no aggregates and only slight size differences were observed between uncoated and coated samples resuspended in water (FIG. 1). However, while uncoated polyplex underwent severe aggregation in buffer, HA-coated polyplex were significantly more stable and preserved their homogeneity with a low PdI (FIG. 1). TEM samples suspended in buffer 20 mM HEPES+150 mM NaCl pH 7.4 were analysed without washing. It was found that HA-coating stabilizes the complexes and prevents aggregation, as shown in FIG. 2 and FIG. 3. DLS and TEM analyses revealed that uncoated polyplexes undergo severe aggregation following resuspension in a buffer with physiological pH and ionic strength, while this aggregation is prevented in the case of HA-coated polyplex. High magnification TEM images revealed that HA-coated polyplexes are surrounded by a corona of HA.

### Example 3

### Production of Coated-Chitosan/ODN Complexes with an Automated Inline Mixing System

Preparation of Nucleic Acid Solution:

A 200 μg/ml ODN (ApoB) solution with trehalose and L-histidine concentrations of 0.5% w/v and 3.5 mM, respectively, was prepared as described in Example 2.

Preparation of Chitosan Solution:

A 225 μg/ml chitosan (Mn 10 kDa, 92% DDA) solution with trehalose and L-histidine concentrations of 0.5% w/v and 3.5 mM, respectively, was prepared as described in Example 2.

Preparation of HA Solution:

A 390 μg/mL HA (Mn=866 kDa) solution with trehalose and L-histidine concentrations of 0.5% w/v and 3.5 mM, respectively, was prepared as described in Example 2.

Automated Production of Complexes:

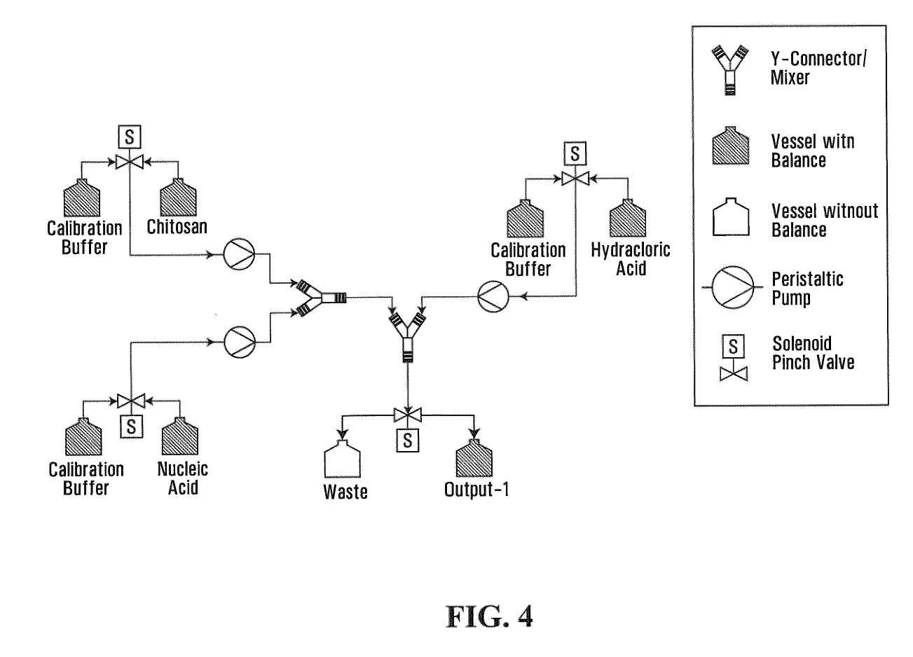

Mixing platform design: The mixing platform is based on three peristaltic pumps that drives CS, NA and HA solutions through a closed set of silicon tubings (ID= 1/16″) and vessels comprising Y-connectors for mixing of the solutions and production of the (FIG. 4). A fast switching pinch valve, located close to the collecting vessel, was used to discard the first 1 mL of the mixture into a waste vessel to ensure homogeneity of the produced nanoparticles. Washing: The vessels, connections, and tubings of the in-line mixing system were washed with detergent (Alconox 1% w/v), then rinsed with double deionized water a few times by pumping water inside the tubings. Calibration: Each pump was computer calibrated prior to mixing. Priming: The system was firstly primed by pumping the solutions (rate=5 mL/min). Priming was done individually for each solution. 1 mL of each solution was used to completely fill the tubings prior to mixing.

Continuous Mixing (MODE 1):

In this mode, two Y-connectors and three pumps were used. For production of complexes, the three pumps were started simultaneously, with a flow rate of 150 mL/min (mixing volumetric ratio of 1 for each component) and were set to dispense 3 mL of each solution. Chitosan and nucleic acid were mixed (to produce chitosan/ODN nanoparticles) in the 1^{st} Y connector. HA was mixed with chitosan/ODN polyplex in a second Y connector located downstream of the 1^{st} one. Polyplexes were produced with N:P:C=2:1.5:1.

Discontinuous Mixing (MODE 2):

In this mode, one Y connector and two pumps were used. For production of complexes, the two pumps were started simultaneously to drive chitosan and ODN solutions toward the Y-connector, with a flow rate of 150 mL/min (mixing volumetric ratio of 1 for the two components). Each pump was controlled to dispense 3 mL of each solution. The produced polyplex solution was kept for 30 minutes at room temperature, then, using the same in-line mixing configuration, 1.5 mL of HA solution were mixed with 3 mL of the CS/ODN polyplex solution using flow rates of 75 and 150 mL/min, respectively. The polyplexes were produced with N:P:C=2:1.5:1.

Manual Preparation of Complexes:

Two samples were also prepared manually for comparison purposes. The first manually mixed sample was prepared as described in Example 2. For the second manually mixed sample, the HA solution was added immediately after mixing CS and ODN, to simulate the continuous in-line mixing method (MODE1). Preparation was done twice for each method. The polyplexes were produced with N:P:C=2:1.5:1.

DLS Measurements:

Samples were analyzed for their size, PDI and zeta potential. 50 μL of each sample was diluted 8× by adding 350 μL of milli-Q water, and further diluted 2× by addition of 400 μL 20 mM NaCl. 400 μL of each solution was added into a DTS1070 cell. For each sample, three consecutive size and zeta potential analyses were done. The instrument was operated in automatic mode. Average values of size, PDI and zeta potential for different samples were calculated.

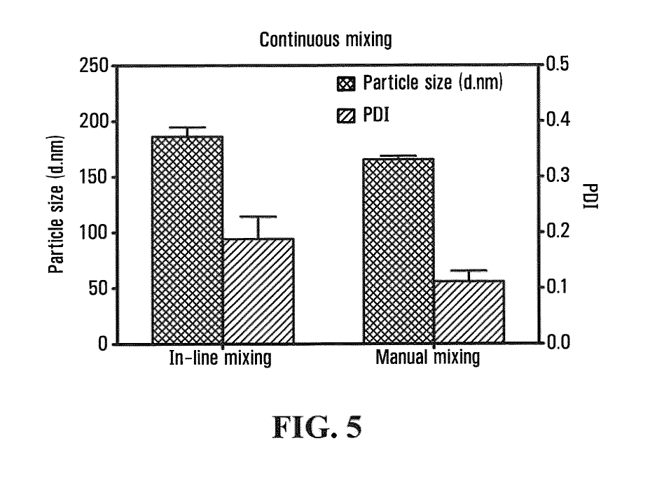

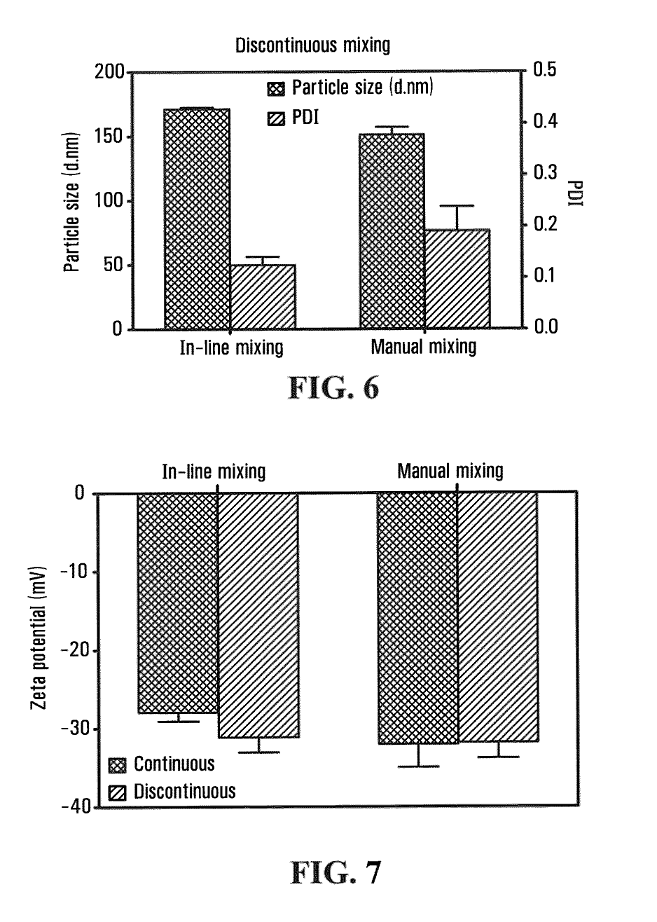

Complexes produced in-line through both continuous and discontinuous mixing methods were not aggregated and had size comparable to that of manually prepared particles (FIG. 5). Particles produced using both continuous and discontinuous in-line mixing methods were homogeneous with low PDIs of 0.18 and 0.12, respectively (FIG. 6). In-line and manually-mixed complexes had a zeta potential of about −30 mV (Figure).

### Example 4

### Stability and Concentration of HA-Coated siRNA/CS Complexes May be Increased Using Freeze-Drying Technique

Preparation of Nucleic Acid Solution:

A 200 μg/ml siRNA (ApoB) solution with trehalose and L-histidine concentrations of 0.5% w/v and 3.5 mM, respectively, was prepared as described in Example 2.

Preparation of Chitosan Solution:

A 225 μg/ml chitosan (Mn 10 kDa, 92% DDA) solution with trehalose and L-histidine concentrations of 0.5% w/v and 3.5 mM, respectively, was prepared as described in Example 2

Preparation of HA solution: A 390 μg/mL HA (Mn=866 kDa) solution with trehalose and L-histidine concentrations of 0.5% w/v and 3.5 mM, respectively, was prepared as described in Example 2.

Preparation of HA Coated Chitosan/siRNA Complexes:

800 μL, of chitosan solution, 800 μL of siRNA solution, and 800 μL of HA solution was mixed manually as described in Example 2. Preparation of complexes was done three times in three different occasions.

Sample Freeze-Drying:

700 μL of each prepared sample of complexes were transferred into clear 2 mL borosilicate serum vials. 2.1 mL of each prepared complex was transferred in three 2 mL serum vials and capped halfway with lyophilization stoppers. Freeze-drying was carried in a Millrock Laboratory Series Freeze-Dryer PC/PLC, using the following cycle: ramped freezing from room temperature to −40° C. in 1 hour, then maintaining isothermal at −40° C. for 2 hours; primary drying for 48 hours at −40° C., at 100 millitorrs; and secondary drying at 100 millitorrs, increasing temperature to 30° C. in 12 hours and then maintaining isothermal at 30° C. for 6 hours. Samples were stoppered, crimped and stored at 4° C. until use. 15 to 30 minutes prior to use, samples were rehydrated by using a volume of RNase free water either equal to their initial volume or reduced.

DLS Measurements:

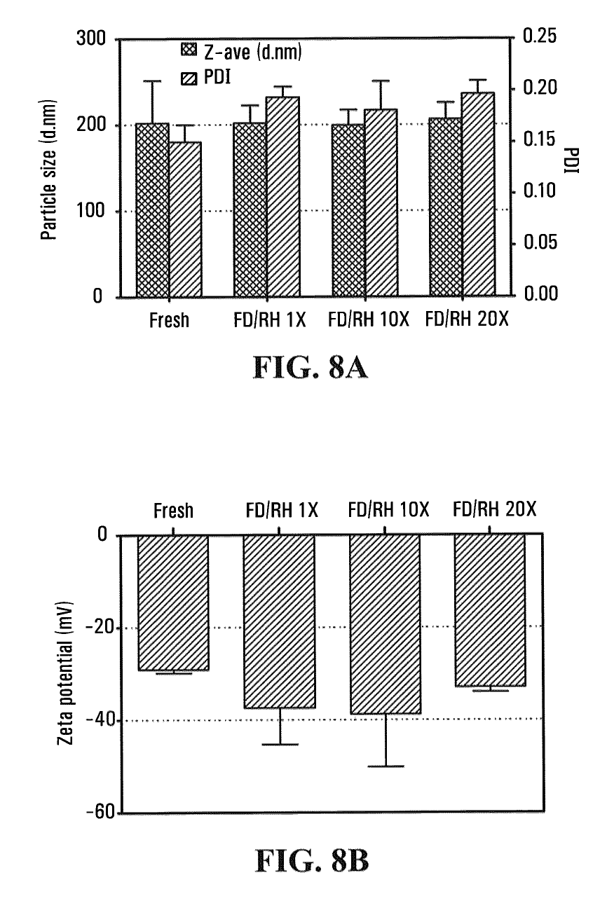

Four samples were analyzed for their size, PDI and zeta potential upon preparation: one freshly prepared and three following a freeze-drying and rehydration to either initial volume or reduced volumes of 70 μL and 35 μL of RNase free water for concentration factor of 10× and 20×, respectively. Upon rehydration, each sample was left untouched for 15 to 30 minutes to stabilize. Then, 10× and 20× rehydrated samples were supplemented with RNase free water to their original volume (700 μL) prior to size and zeta potential analysis. Each sample was diluted 16× for DLS analysis: 50 μL of each sample was diluted 8× by adding 350 μL of RNase free water, and further diluted 2× by addition of 400 μL of 20 mM NaCl. 400 μL of each solution was added into a DTS1070 cell. Analysis was then performed as described in Example 3. Nanoparticles formulated in 0.5% (w/v) trehalose dihydrate and 3.5 mM L-histidine could be freeze-dried to reach the final concentration factor of 20× without seeing particle aggregation. As compared to freshly prepared particles, freeze-dried complexes have almost the same Z-averages (FIG. 8A). PdI values only slightly increased following freeze-drying and rehydration, from 0.15, when freshly prepared, to between 0.18 and 0.20 (FIG. 8A). Freshly prepared nanoparticles had an average zeta potential of −29 mV; freeze-dried and rehydrated compositions had zeta potentials of −38 to −32 mV (FIG. 8B).

### Example 5

### Effects of Chitosan Formulations on Erythrocyte Hemolysis and Hemagglutination

Preparation of Freeze-Dried Chitosan Formulations Containing No HA:

Chitosans (Mₙ 10 kDa and 92% DDA, Mₙ 40 kDa and 92% DDA; Mₙ 120 kDa and 92% DDA) were dissolved in HCl to obtain a stock concentration of 5 mg/mL. Chitosans were further diluted with nuclease-free water to obtain solutions with chitosan concentrations of 2.25 mg/mL (for all chitosans) or 1.125 mg/mL (for chitosan Mₙ 120 kDa and 92% DDA only). Nucleic acid stock solutions were prepared by diluting with nuclease-free water at 800 μg/mL concentration. Histidine stock was prepared at 28 mM pH 6.5 and trehalose stock was prepared at 4% w/v. Chitosan working solutions were prepared by mixing equal volumes (2.7 mL) of dilute chitosan solutions (2.25 mg/mL or 1.125 mg/mL), nuclease-free water, stock histidine solution and stock trehalose solution. Nucleic acid working solutions were prepared by mixing equal volumes (2.7 mL) of nucleic acids (800 μg/mL), nuclease-free water, stock histidine solution and stock trehalose solution. Complexes were prepared manually by mixing equal volumes (1 mL at a time) of chitosan working solutions and nucleic acid working solutions and incubating for 15 minutes. Nucleic acids were either ApoB ODN or ApoB siRNA. Formulations containing chitosan but no nucleic acids were prepared as described above except that the nucleic acid component was replaced with nuclease-free water. Complexes were aliquoted into glass vials and freeze-dried as described in Example 4. Concentrations of the different components present in the freeze-dried cakes are described in Table 2.

TABLE 2
Freeze-dried formulations containing chitosan and no HA were prepared for
hemocompatibility testing.

| | CS conc | siRNA or ODN | Trehalose | Histidine | HA |
|---|---|---|---|---|---|
| CS | (w/v) | conc (w/v) | (w/v) | (w/v) | (w/v) |
| 92-10, 92-40, 92-120 | 0.28 mg/mL | 0 mg/mL | 10 mg/mL | 1.09 mg/mL | 0 mg/mL |
| 92-120 | 0.14 mg/mL | 0 mg/mL | 10 mg/mL | 1.09 mg/mL | 0 mg/mL |
| 92-10, 92-40 | 0.28 mg/mL | 0.1 mg/mL | 10 mg/mL | 1.09 mg/mL | 0 mg/mL |
| 92-120 | 0.14 mg/mL | 0.05 mg/mL | 10 mg/mL | 1.09 mg/mL | 0 mg/mL |

Preparation of Freeze-Dried Chitosan/HA/Nucleic Acid Formulations:

Chitosan Mₙ 10 kDa and 92% DDA was dissolved in HCl to obtain a stock concentration of 5 mg/mL. HA stock solution was prepared at a concentration of 2.5 mg/mL. Apo B ODN stock solution was prepared by diluting with nuclease-free water at 1 mg/mL. Histidine stock was prepared at 28 mM pH 6.5 and trehalose stock was prepared at 4% w/v. Chitosan working solution was prepared by mixing 144 μL chitosan solution (5 mg/mL), 2256 μL nuclease-free water, 400 μL stock histidine solution and 400 μL stock trehalose solution. Apo B ODN working solution was prepared by mixing 640 μL of ODN (1 mg/mL), 1760 pit nuclease-free water, 400 μL stock histidine solution and 400 μL stock trehalose solution. HA working solution was prepared by mixing 512 μL HA stock solution (2.5 mg/mL), 1888 μL nuclease-free water, 400 μL stock histidine solution and 400 μL stock trehalose solution. Complexes were prepared by manually mixing 1 mL of chitosan working solution and 1 mL of Apo B ODN working solution and incubating for 30 minutes. Then, 1 mL of HA working solution was added and left to incubate for another 30 minutes. Complexes were aliquoted into glass vials and freeze-dried as described in Example 4. Concentrations of the different components present in the freeze-dried cakes are described in Table 3.

Preparation of Freeze-Dried Chitosan/HA Formulations without Nucleic Acids:

Chitosan Mₙ 10 kDa and 92% DDA was dissolved in HCl to obtain a stock concentration of 5 mg/mL. HA stock solution was prepared at a concentration of 2.5 mg/mL. Apo B ODN stock solution was prepared by diluting with nuclease-free water at 1 mg/mL. Histidine stock was prepared at 28 mM pH 6.5 and trehalose stock was prepared at 4% w/v. Chitosan working solution was prepared by mixing 144 μL chitosan solution (5 mg/mL), 2256 μL nuclease-free water, 400 μL stock histidine solution and 400 μL stock trehalose solution. To replace the Apo B ODN, a working water solution was prepared by mixing 2400 μL nuclease-free water with 400 μL stock histidine solution and 400 μL stock trehalose solution. HA working solution was prepared by mixing 835 μL HA stock solution (2.5 mg/mL), 1565 μL nuclease-free water, 400 μL stock histidine solution and 400 μL stock trehalose solution. Formulations were prepared by manually mixing 1 mL of chitosan working solution, 1 mL of water working solution and 1 mL of HA working solution. Formulations were aliquoted into glass vials and freeze-dried as described in Example 4. Concentrations of the different components present in the freeze-dried cakes are described in Table 3.

TABLE 3
Freeze-dried formulations containing chitosan and HA were prepared for
hemocompatibility testing.

| CS | CS conc (w/v) | siRNA conc (w/v) | Trehalose (w/v) | Histidine (w/v) | HA (w/v) |
|---|---|---|---|---|---|
| 92-10 | 0.075 mg/mL | 0 mg/mL | 4.9 mg/mL | 0.55 mg/mL | 0.22 mg/mL |
| 92-10 | 0.075 mg/mL | 0.066 mg/mL | 4.9 mg/mL | 0.55 mg/mL | 0.13 mg/mL |

Isolation and Dilution of Human Blood:

Na citrate anti-coagulated blood was collected from human donors. The cyanmethemoglobin colorimetric assay (Zwart, vanAssendelft et al. 1996) was used to quantify the plasma free hemoglobin and total blood hemoglobin of the samples. The blood was diluted with PBS to adjust total blood hemoglobin concentration to 10±2 mg/mL.

Reconstitution and Dilution of Chitosan and Chitosan/HA Formulations:

The cakes containing chitosan and no HA (Table 2) were reconstituted in nuclease-free water using a 7.5× concentration factor and the reconstituted chitosan formulations were further diluted with PBS in order to obtain the final concentrations indicated in Table 4.

TABLE 4
Dilution of chitosan formulations.

| | | | | | | | ODN/ | | |
|---|---|---|---|---|---|---|---|---|---|
| | | CS conc | | Trehalose | | Histidine | siRNA | | HA |
| CS | Dilution | (w/v) | | (w/v) | | (w/v) | (w/v) | | (w/v) |
| 92-10 | D1 | 2.1 | mg/mL | 75 | mg/mL | 8.150 mg/mL | 0 | mg/mL | 0 mg/mL |
| 92-40 | D2 | 1.491 | mg/mL | 53.25 | mg/mL | 5.787 mg/mL | 0 | mg/mL | 0 mg/mL |
| 92-120 | D3 | 0.756 | mg/mL | 27 | mg/mL | 2.934 mg/mL | 0 | mg/mL | 0 mg/mL |
| | D4 | 0.294 | mg/mL | 10.5 | mg/mL | 1.141 mg/mL | 0 | mg/mL | 0 mg/mL |
| | D5 | 0.147 | mg/mL | 5.25 | mg/mL | 0.571 mg/mL | 0 | mg/mL | 0 mg/mL |
| | D6 | 0.029 | mg/mL | 1.050 | mg/mL | 0.114 mg/mL | 0 | mg/mL | 0 mg/mL |
| 92-120 | D1 | 1.05 | mg/mL | 75 | mg/mL | 8.150 mg/mL | 0 | mg/mL | 0 mg/mL |
| | D2 | 0.746 | mg/mL | 53.25 | mg/mL | 5.787 mg/mL | 0 | mg/mL | 0 mg/mL |
| | D3 | 0.378 | mg/mL | 27 | mg/mL | 2.934 mg/mL | 0 | mg/mL | 0 mg/mL |
| | D4 | 0.147 | mg/mL | 10.5 | mg/mL | 1.141 mg/mL | 0 | mg/mL | 0 mg/mL |
| | D5 | 0.074 | mg/mL | 5.25 | mg/mL | 0.571 mg/mL | 0 | mg/mL | 0 mg/mL |
| | D6 | 0.015 | mg/mL | 1.050 | mg/mL | 0.114 mg/mL | 0 | mg/mL | 0 mg/mL |
| 92-10 | D1 | 2.1 | mg/mL | 75 | mg/mL | 8.150 mg/mL | 0.75 | mg/mL | 0 mg/mL |
| 92-40 | D2 | 1.491 | mg/mL | 53.25 | mg/mL | 5.787 mg/mL | 0.533 | mg/mL | 0 mg/mL |
| | D3 | 0.756 | mg/mL | 27 | mg/mL | 2.934 mg/mL | 0.270 | mg/mL | 0 mg/mL |
| | D4 | 0.294 | mg/mL | 10.5 | mg/mL | 1.141 mg/mL | 0.105 | mg/mL | 0 mg/mL |
| | D5 | 0.147 | mg/mL | 5.25 | mg/mL | 0.571 mg/mL | 0.053 | mg/mL | 0 mg/mL |
| | D6 | 0.029 | mg/mL | 1.050 | mg/mL | 0.114 mg/mL | 0.011 | mg/mL | 0 mg/mL |
| 92-120 | D1 | 1.05 | mg/mL | 75 | mg/mL | 8.150 mg/mL | 0.375 | mg/mL | 0 mg/mL |
| | D2 | 0.746 | mg/mL | 53.25 | mg/mL | 5.787 mg/mL | 0.266 | mg/mL | 0 mg/mL |
| | D3 | 0.378 | mg/mL | 27 | mg/mL | 2.934 mg/mL | 0.135 | mg/mL | 0 mg/mL |
| | D4 | 0.147 | mg/mL | 10.5 | mg/mL | 1.141 mg/mL | 0.053 | mg/mL | 0 mg/mL |
| | D5 | 0.074 | mg/mL | 5.25 | mg/mL | 0.571 mg/mL | 0.026 | mg/mL | 0 mg/mL |
| | D6 | 0.015 | mg/mL | 1.050 | mg/mL | 0.114 mg/mL | 0.005 | mg/mL | 0 mg/mL |

The cakes containing chitosan and HA were reconstituted in nuclease-free water using a 11.4× concentration factor and the reconstituted chitosan/HA formulations were further diluted with PBS in order to obtain the final concentrations indicated in Table 5.

TABLE 5
Dilution of chitosan/HA formulations.

| | | CS conc | | Trehalose | | Histidine | | ODN conc | | HA conc |
|---|---|---|---|---|---|---|---|---|---|---|
| CS | Dilution | (w/v) | | (w/v) | | (w/v) | | (w/v) | | (w/v) |
| 92-10 | D1 | 0.86 | mg/mL | 56 | mg/mL | 6.28 | mg/mL | 0 | mg/mL | 2.49 mg/mL |
| | D2 | 0.31 | mg/mL | 20 | mg/mL | 2.26 | mg/mL | 0 | mg/mL | 0.90 mg/mL |
| | D3 | 0.12 | mg/mL | 7.8 | mg/mL | 0.88 | mg/mL | 0 | mg/mL | 0.35 mg/mL |
| | D4 | 0.012 | mg/mL | 0.75 | mg/mL | 0.084 | mg/mL | 0 | mg/mL | 0.03 mg/mL |
| 92-10 | D1 | 0.86 | mg/mL | 56 | mg/mL | 6.28 | mg/mL | 0.75 | mg/mL | 1.52 mg/mL |
| | D2 | 0.31 | mg/mL | 20 | mg/mL | 2.26 | mg/mL | 0.270 | mg/mL | 0.55 mg/mL |
| | D3 | 0.12 | mg/mL | 7.8 | mg/mL | 0.88 | mg/mL | 0.105 | mg/mL | 0.21 mg/mL |
| | D4 | 0.012 | mg/mL | 0.75 | mg/mL | 0.084 | mg/mL | 0.011 | mg/mL | 0.02 mg/mL |

Hemolysis Testing:

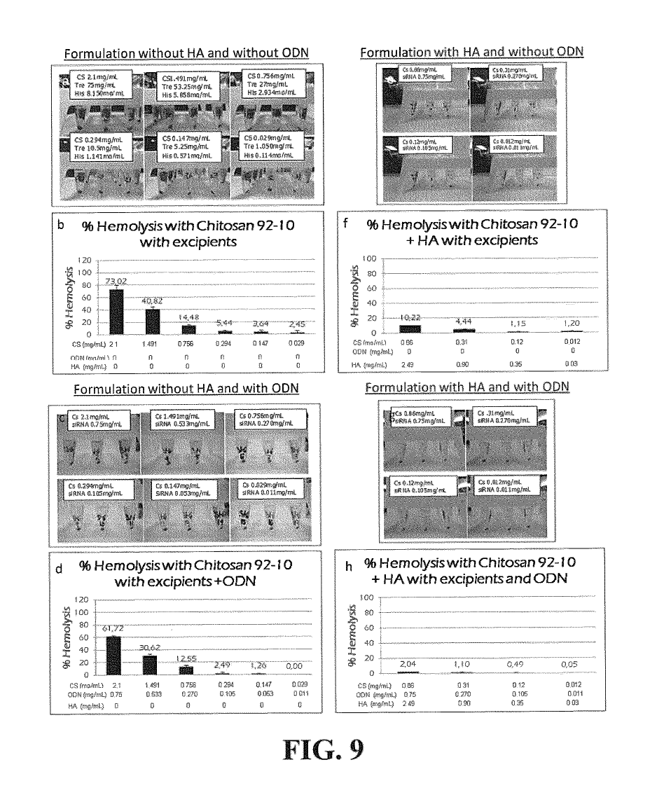

100 μL of each chitosan and chitosan/HA dilution was pipetted into Eppendorf tubes. 700 μL of PBS was added to each tube. 100 μL of the diluted blood sample was added to each tube. The tubes were incubated for 3 hours in a water bath set at 37° C. The tubes were centrifuged for 15 min at 800 g and photo documented. The cyanmethemoglobin colorimetric assay was used to quantify the hemoglobin in the supernatant and % erythrocyte hemolysis quantified Erythrocyte haemolysis was induced by the chitosan formulations at the highest CS concentrations and haemolysis decreased as the formulations were diluted (FIGS. 9A and 9B). The formulations containing chitosan and hyaluronic acid (HA) did not induce much erythrocyte haemolysis (FIGS. 9E and 9F). The addition of HA protected the cells from haemolysis. Adding nucleic acid to the formulations decreased erythrocyte haemolysis (FIGS. 9C, 9D, 9G and 9H). Free chitosan appears to interact with blood components leading to lysis of the cells.

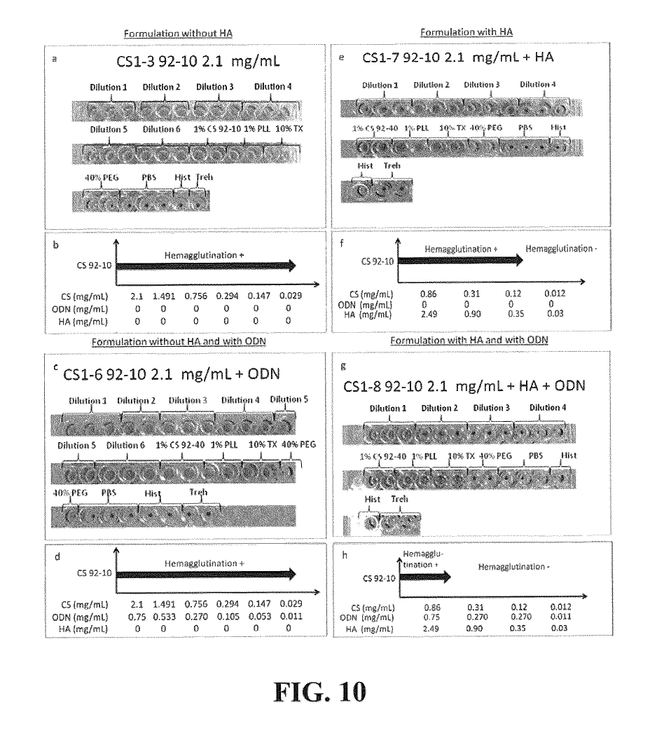

Hemagglutination testing: 100 μL of each chitosan and chitosan/HA dilution was pipetted into Eppendorf tubes. 700 μL of PBS was added to each tube. 100 μL of the diluted blood sample was added to each tube. 200 μL each mixture was pipetted into round-bottomed 96-well plates and incubated for 3 hours in a Pasteur oven set at 37° C. The plates were photodocumented after 3 hours. All freeze-dried formulations that contained chitosan without HA induced hemagglutination, whether in absence or in presence of nucleic acid (FIGS. 10A to 10D). Hemagglutination was induced even at the most diluted chitosan concentrations. Only the most concentrated chitosan/HA formulations induced erythrocyte hemagglutination (FIGS. 10E and 10F). Adding HA to the formulations prevented erythrocyte hemagglutination. Adding nucleic acid to the chitosan/HA formulations decreased erythrocyte hemagglutination (FIGS. 10G and 10H). Free chitosan appears to interact with erythrocytes leading to agglutination.

### Example 6

### Administration of Chitosan Polyplexes

Preparation of Uncoated Chitosan/siRNA Complexes:

CS-siRNA (ApoB) polyplexes were prepared with a N:P ratio of 2 or 5 in presence of trehalose (0.5% w/v) and L-histidine (3.5 mM). Chitosans 92-10, 92-40 and 92-120 were used. Polyplexes were prepared manually as described above in Example 2. Final concentration of siRNA was 0.1 mg/mL or 0.05 mg/mL for polyplex prepared with CS 92-10 and CS 92-40 or 92-120, respectively. Polyplexes were freeze-dried as described in Example 4.

Preparation of HA Coated Chitosan/siRNA Complexes:

HA-coated polyplexes were prepared manually using HA (Mn=866 kDa), CS 92-10 and siRNA ApoB with a N:P:C ratio of 2:1:1.5, as described in Example 2. Polyplexes were freeze-dried as described in Example 4.

Preparation of Invivofectamine® 2.0-siRNA Complexes:

Invivofectamine (commercial product with low toxicity profile with effective siRNA delivery) nanoparticles preparation was performed under sterile condition and according to the manufacturer instructions. Briefly, the ApoB siRNA solution was prepared at 1.5 mg/ml using the complexation buffer. Invivofectamine®2.0 Reagent was thawed and added to the siRNA solution with thorough mixing. In order to remove the toxicity from salts, diafiltration was done using Amicon® Ultra-15 column. The retentate containing the Invivofectamine®2.0-siRNA complexes was collected and stored at 4° C. until injection. Before injection, the animal weights were measured and accordingly, the volume of each injection was calculated.

Rehydration of Freeze-Dried Samples:

Prior to injection, the freeze-dried samples were rehydrated with nuclease-free water using a reduced reconstitution volume (20× concentration factor). After reconstitution, trehalose 10% w/v—histidine 70 mM (pH 6.5) was added to reach the required siRNA concentration such that a mouse would receive appropriate dosage of 7, 5, 3.5, 2.5 or 1 mg/kg by injecting 9.5 μL/gram of the animal.

Injection of Test/Control Articles:

C57BL/6 mice were intravenously injected with uncoated CS/siRNA, HA-coated CS-siRNA NPs, Invivofeactamine and other controls shown in Table 6 below.

TABLE 6
Test/Control Articles injected to mice C57bl/6.

| | | | | | Dose of |
|---|---|---|---|---|---|
| | | | | | siRNA |
| | | | | | (or LPS or |
| | | | | | Poly I:C) |
| | | | | | injected |
| Group | N = | Delivery System | N:P:C | Route | (mg/kg) |
| 1 | 2 | LPS | — | IV | 1 |
| | 2 | LPS | | IV | 10 |
| 2 | 3 | Poly (LC) | — | IP | 20 |
| 3 | 3 | PBS | — | IV | — |
| 4 | 3 | Invivofectamine/siRNA ApoB | — | IV | 7 |
| 5 | 3 | Naked siRNA | — | IV | 7 |
| 6 | 3 | 92-10 Chitosan/siRNA ApoB | 5:1.0 | IV | 7 |
| 7 | 1 | 92-40 Chitosan/siRNA ApoB | 5:1.0 | IV | 7 |
| 8 | 3 | 92-120 Chitosan/siRNA | 5:1.0 | IV | 3.5 |
| | | ApoSB | | | |
| 9 | 1 | 92-10 Chitosan/siRNA ApoB | 5:1.0 | IV | 1 |
| 10 | 1 | 92-10 Chitosan/siRNA ApoB | 2:1:0 | IV | 1 |
| 11 | 1 | 92-10 HA-Chitosan/siRNA | 2:1:1.5 | IV | 7 |
| | | ApoB | | | |
| 12 | 1 | 92-10 HA-Chitosan/siRNA | 2:1:1.5 | IV | 5 |
| | | ApoB | | | |
| 13 | 1 | 92-10 HA-Chitosan/siRNA | 2:1:1.5 | IV | 2.5 |
| | | ApoB | | | |

Clinical Signs Scoring System

An overall score ranging from 3 to 0 was assigned:
Score of 3: no sign of distress
Score of 2: signs of discomfort
Score of 1: some signs of distress
Score of 0: important signs of distress

Control Clinical Signs:

No signs of clinical toxicity (score of 3) was observed for PBS, LPS (1 mg/kg), Poly (I:C) (20 mg/kg) and Invivofectamine NPs. Mild signs of clinical toxicity with score of 2 was noticed for LPS when injected at a dose of 10 mg/kg.

Uncoated NPs Clinical Signs:

No signs of clinical toxicity (score of 3) were associated with uncoated chitosan nanoparticles at 1 mg/kg. Mild signs of clinical toxicity (score 2) were observed for the uncoated chitosan nanoparticles at 2.5 mg/kg (mild signs of distress for short period of time post-injection, ˜20 minutes). Moderate signs of clinical toxicity with scores 1-2 were observed for uncoated chitosan nanoparticles at 5 mg/kg (signs of distress for ˜45 minutes post injection). Severe signs of clinical toxicity with scores 0-1 were observed for uncoated chitosan-siRNA at 7 mg/kg. In addition, distress appears to increase with chitosan molecular weight.

HA-Coated NPs Clinical Signs:

No signs of clinical toxicity (score of 3) were noticed for HA coated chitosan nanoparticles at 2.5 mg/kg and below. Mild signs of clinical toxicity with a score of 2 were observed for HA coated CS NPs at 5 and 7 mg/kg (mild signs of distress for short period of time post-injection, ˜10 and 30 minutes, respectively). According to clinical signs observed in C57b1-6 mice following CS-siRNA nanoparticles injection, the HA coated chitosan system is well tolerated. Uncoated CS-siRNA nanoparticles are more toxic but showed no signs of toxicity at a dose of 1 mg/kg.

### Example 7

### Evaluation of In Vivo Toxicity and Inflammation of Chitosan-siRNA Complex and HA Coated Chitosan-siRNA Systems

The purpose of this study was to evaluate the toxicity and inflammation of chitosan-siRNA complex and HA-coated chitosan-siRNA complex systems in vivo.

Rationale for Selecting siRNA Sequences and Pro-Inflammatory Controls:

siRNA sequences were chosen from the art as they were previously demonstrated to induce inflammatory cytokines depending on structure, composition and type of chemical modification. The siRNA ApoB (native) was shown to induce high TNF and INF-α levels following its administration with lipid based systems (LNPs) (Judge, Bola et al. 2006). Therefore this sequence was selected as a pro-inflammatory model to demonstrate the safety of the delivery system. It is generally recognized in the art that chemical modification of siRNAs abrogate immune induction. The 2′-O methylated sequence, namely the siRNA ApoB (2′OMe U(S)), was chosen because it has been shown to reduce the cytokine induction vs native form (Judge, Bola et al. 2006). LPS, from Escherichia coli, is a potent inducer of inflammatory cytokines related to hepatotoxicity and can be measured by changes in the aspartate aminotransferase (AST), alanine aminotransferase (ALT) activities and total bilirubin levels in serum, and hepatic glutathione contents.

siRNA Sequences Used in the Study

| siRNA ApoB (native): | |
|---|---|
| Sense: | 5′-GUCAUCACACUGAAUACCAAU |
| Antisense: | 5′-AUUGGUAUUCAGUGUGAUGACAC |
| siRNA ApoB (2′OMe U(S)): | |
| Sense: | 5′-GmUCAmUCACACmUGAAmUACCAAmU |
| Antisense: | 5′-PAUUGGUAUUCAGUGUGAUGACAC |
| | (m = 2′O methylation) |

Preparation of Uncoated Chitosan/siRNA Complexes:

CS/siRNA polyplexes were prepared using the automated in-line mixing system, as described in Example 3 but without HA. Fully characterized, CS 92-10 was complexed with either the native or the 2′O methylated form of ApoB at an N:P ratios of 2 and 5. Polyplexes were freeze-dried as described in Example 4.

Preparation of HA Coated Chitosan/siRNA Complexes:

The HA-coated polyplexes were prepared using the automated in-line mixing system using discontinuous mixing method, as described in Example 3. HA-coated polyplexes were prepared using HA 866 kDa, CS 92-10 and the native and 2′-O methylated forms of siRNA ApoB with N:P:C=2:1:1.5. Polyplexes were freeze-dried as described in Example 4.

Preparation of Invivofectamine®2.0-siRNA Complexes:

Invivofectamine nanoparticles were prepared as described in Example 6.

Injection of Test/Control Articles and Design of Study:

Chitosan siRNA based polyplexes were rehydrated in water and further diluted in excipients in order to reach target doses. CD1 mice were intravenously injected into the tail vein with the test and control articles shown in Table 7. Clinical toxicity or clinical signs were assessed using a hybrid scoring system taking into account the general aspect score, the provoked behaviour score and the mouse grimace scale. Unless otherwise stated, clinical signs were observed every 10 minutes for 4 hours post injection and at euthanasia. A group of 7 mice/treatment were injected expect for group 20 where 5 animals were injected with excipients.

TABLE 7
Test/Control Articles injected to mice CD1.

| | | | | siRNA |
|---|---|---|---|---|
| | | | | dose to be |
| | | | | injected in |
| | | | | animal |
| Group | N = | Control/Test Article description | N:P:C | (mg/kg) |
| 1 | 7 | PBS | — | — |
| 2 | 7 | LPS | — | TBD |
| | | | | (10 mg/kg), |
| 3 | | Chitosan/ApoB (native)-siRNA | 5:1:0 | 2.5 |
| 4 | 7 | Chitosan/ApoB (native)-siRNA | 5:1:0 | 1 |
| 5 | 7 | Chitosan/ApoB 2′OMe siRNA | 5:1:0 | 2.5 |
| 6 | 7 | Chitosan/ApoB (native)-siRNA | 2:1:0 | 5 |
| 7 | 7 | Chitosan/ApoB (native)-siRNA | 2:1:0 | 2.5 |
| 8 | 7 | Chitosan/ApoB (native)-siRNA | 2:1:0 | 1 |
| 9 | 7 | Chitosan/ApoB (2OMe)-siRNA | 2:1:0 | Max dose |
| | | | | tolerated |
| | | | | from |
| | | | | groups |
| | | | | above |
| 10 | 7 | Chitosan-HA/ApoB (native)-siRNA | 2:1:1.5 | 8 |
| 11 | 7 | Chitosan-HA/ApoB (native)-siRNA | 2:1:1.5 | 5 |
| 12 | 7 | Chitosan-HA/ApoB (native)-siRNA | 2:1:1.5 | 2.5 |
| 13 | 7 | Chitosan-HA/ApoB (native)-siRNA | 2:1:1.5 | 1 |
| 14 | 7 | Chitosan-HA/ApoB (2OMe)-siRNA | 2:1:1.5 | 8 |
| 15 | 7 | Invivofectamine/ApoB (native)-siRNA | — | 8 |
| 16 | 7 | Invivofectamine/ApoB (native)-siRNA | — | 5 |
| 17 | 7 | Invivofectamine/ApoB (native)-siRNA | — | 2.5 |
| 18 | 7 | Invivofectamine/ApoB (native)-siRNA | — | 1 |
| 19 | 7 | Invivofectamine/ApoB (2OMe)-siRNA | — | 8 |
| 20 | 5 | Excipients | — | Dose |
| | | | | equivalent |
| | | | | to the |
| | | | | concen- |
| | | | | tration |
| | | | | in the |
| | | | | 8 mg/kg |
| | | | | dose |

Cytokine Induction and Blood Biochemical Parameters.

A blood volume between 100-200 μL was collected via mandibular puncture 4 hours post injection. Collected blood was allowed to clot for 15 minutes at room temperature and serum separated using a benchtop centrifuge at 10000 rpm for 5 minutes at 4° C. Serum was immediately stored at −80° C. until cytokine analysis. The level of inflammatory serum cytokines, for example, but not limited to, IL-6, TNFα, IFNα, IL-1α, KC and IFN were analyzed by using Bio-Plex multiplex system and ELISA. Two mice per group were sacrificed 4 hours post injection using cardiac puncture and total circulating blood collection. Total circulating blood volume was also serum separated using BD vacutainer gold tubes. The remaining 5 mice/group were sacrificed 24 hours post injection and blood sent to IDEXX laboratories for clinical chemistry and hematological analysis. Measured clinical chemistry parameters included: alanine transaminase (ALT), aspartate aminotransferase (AST), alkaline phosphatase (ALP), Gamma-glutamyl transpeptidase (GGT), albumin and total bilirubin. In addition, nephrotoxicity will also be evaluated based on creatinine, blood urea nitrogen (BUN), glucose, sodium and potassium in the serum. Hematological parameters included and were not limited to: WBC count, red blood cell (RBC) count, hemoglobin (HGB), red blood cell specific volume (HCT), mean corpuscular volume (MCV), mean corpuscular hemoglobin (MCH), mean corpuscular hemoglobin concentration (MCHC), red cell distribution width (RDW-CV), platelet (PLT), platelet distribution width (PDW-CV), mean platelet volume (MPV) and plateletcrit (PCT).

Histology:

Following blood collection, lungs, liver, kidney, spleen, intestine and heart were collected and weighed to understand the treatment related organ weight changes. Subsequently, organs were longitudinally dissected into halves. The first half of each organ was fixed in 10% neutral buffered formalin (NBF) then paraffin embedded for histopathological analysis. The second half of the organs was flash frozen in liquid nitrogen and processed for molecular analysis i.e. northern blotting, PNA assay.

Results:

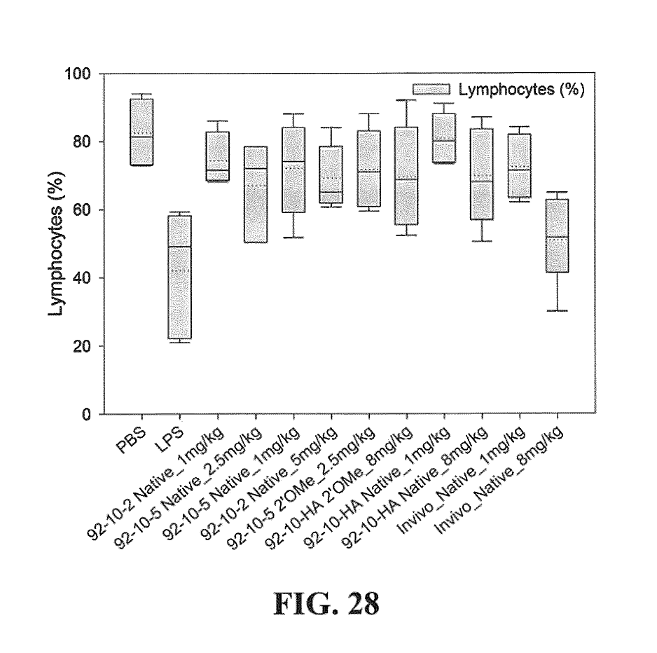

Clinical signs: InvivoFectamine NPs display no signs of clinical toxicity when administered IV at doses up to 8 mg/kg. The LPS control show acute clinical signs with a score of 2-3 for a period of 4 hours followed by an improvement of clinical signs (No death occurred). Uncoated chitosan-siRNA nanoparticles prepared at N:P 5, display no signs of clinical toxicity at doses up to 2.5 mg/kg. Above this dose, clinical signs were observed in a dose dependent manner (data not shown). For the HA coated chitosan-siRNA NPs, no clinical signs were observed up to 5 mg/kg. At a higher dose of 8 mg/kg, very mild clinical signs for the PKBS score were observed for a period of 15-20 minutes. During this period of time, mice seemed to be responding to stimulus with a longer than expected reaction time. The reaction time was around 3-5 seconds instead of 1-3 seconds. No signs for the GAS and Mogul scores were observed at this doses indicating the absence of clinical toxicity (FIG. 28). Table 8 indicates the clinical signs assessed for a period of 4 h following IV administration of siRNA/CS complexes and lipid nanoparticles (Invivofectamine) in CD1 mice.

TABLE 8
Shows clinical signs assessed for a period of 4 h following
IV administration of siRNA/CS complexes and lipid nanoparticles
(Invivofectamine) in CD1 mice.

| Doses | InvivoFectamine 2.0 | | Uncoated | | HA-coated | |
|---|---|---|---|---|---|---|
| (mg/kg) | Score | Freq. | Score | Freq. | Score | Freq. |
| 1 | 0 | 7/7 | 0 | 7/7 | 0 | 7/7 |
| 2.5 | 0 | 7/7 | 1 | 3/7 | 0 | 7/7 |
| 8 | 0 | 7/7 | N/A | 0/0 | 1 | 7/7 |

Score 0: Absence of clinical signs, 0 GAS, 0 PKBS and 0 Mogul scale
Score 1: Absence of clinical signs, 0 GAS, 1 PKBS and 0 Mogul scale
Score 2: Mild clinical signs, 1-2 GAS, 1-2 PKBS and 1-2 Mogul scale
Score 3: Severe clinical signs, 2-3 GAS, 2-3 PKBS and 2-3 Mogul scale

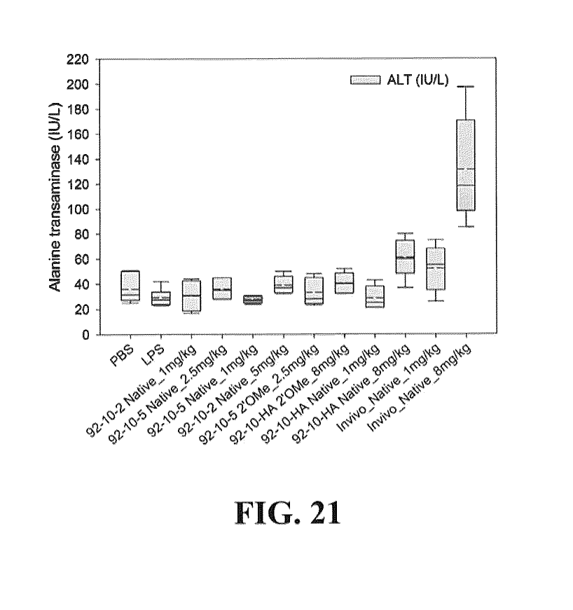

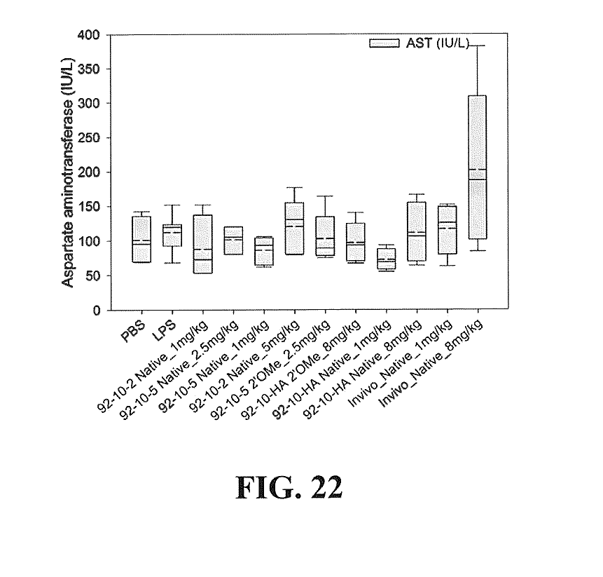

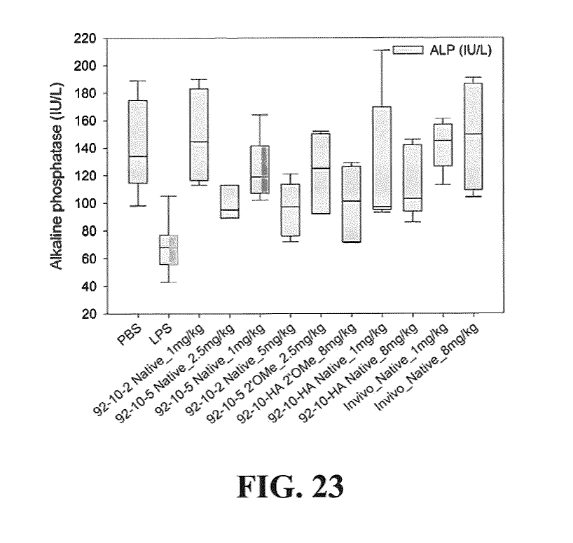

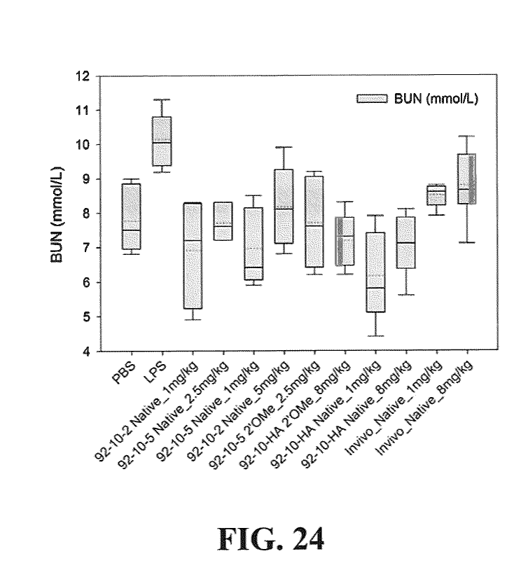

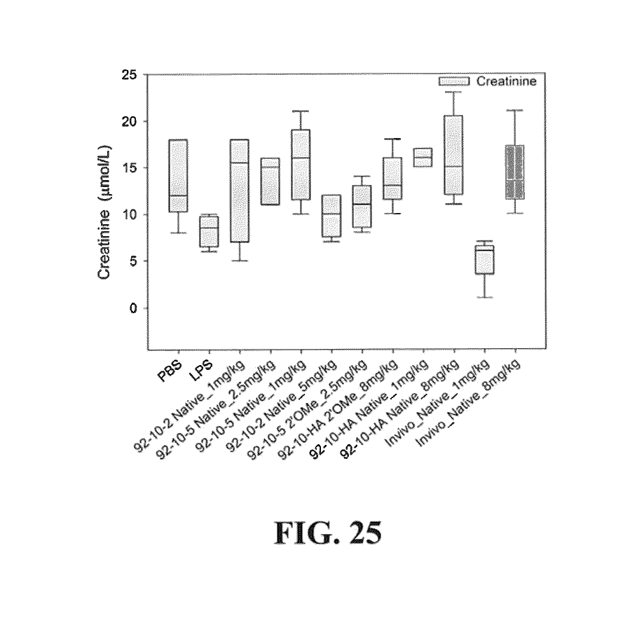

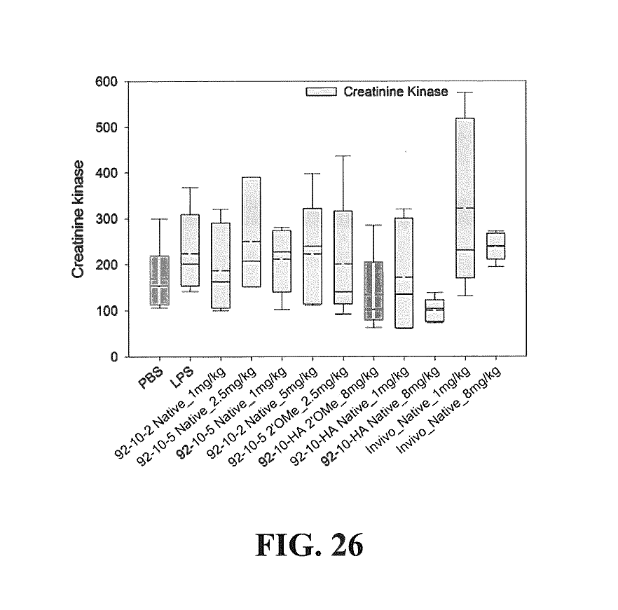

Clinical chemistry: Liver markers of toxicity such as alanine transaminase (ALT), Aspartate aminostransferase (AST) and alkaline phosphatase (ALP) show no hepatic toxicity following injection of chitosan-siRNA complexes (FIGS. 21, 22 and 23). LNPs induce liver toxicity at doses of 8 mg/kg (FIGS. 21, 22 and 23). Although chitosan-siRNA complexes accumulate in the kidneys, there was no change in clinical markers of toxicity associated with kidney such as BUN or creatinine (FIGS. 24 and 25). LNPs show an absence of renal toxicity (FIGS. 24 and 25). Levels of creatinine kinase, a marker of muscle toxicity and injury, did not show any statistical difference with the PBS injected control indicating the absence of muscle related toxicities i.e. smooth, striated or heart muscles. The above results indicate that chitosan-siRNA complexes—uncoated and HA coated—are safe following intravenous injection.

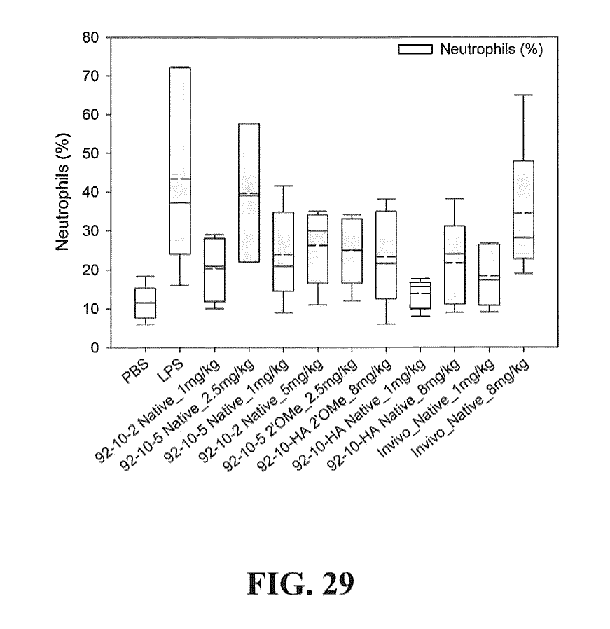

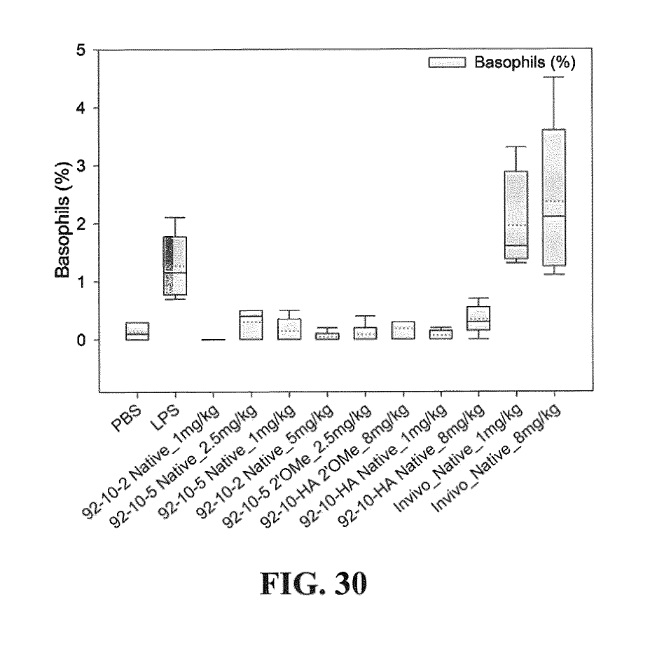

Hematological markers: LNPs induced lymphopenia and increase in basophils and neutrophils content (FIGS. 29, 30 and 31). The change was statistically significant compared to PBS control (FIGS. 29, 30 and 31). The level of lymphopenia and induction of basophils/neutrophils was similar to the LPS injected control indicating acute hematological toxicity and a probable inflammation. Chitosan-siRNA complexes did not change any hematological parameters and were comparable to the PBS control (FIGS. 29, 30 and 31).

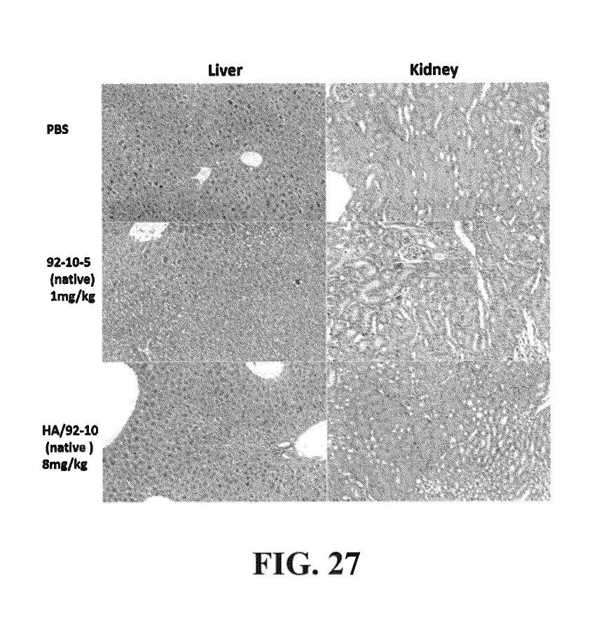

Histology: There was no observed histopathological toxicity with chitosan-siRNA nanoparticles. There was no immune cell infiltration or changes in morphological structures—microscopic structures—as compared to PBS (FIG. 27). LNPs induced an acute immune infiltration in the liver at high doses of 8 mg/kg (data not shown).

Uncoated and HA-coated compositions thus appear to be safe and appear not to induce any observed clinical signs, hematological, clinical chemistry or histopathological changes.

### Example 8

### Biodistribution of HA-Coated Nanoparticles

Preparation and Injection of NPs Solution:

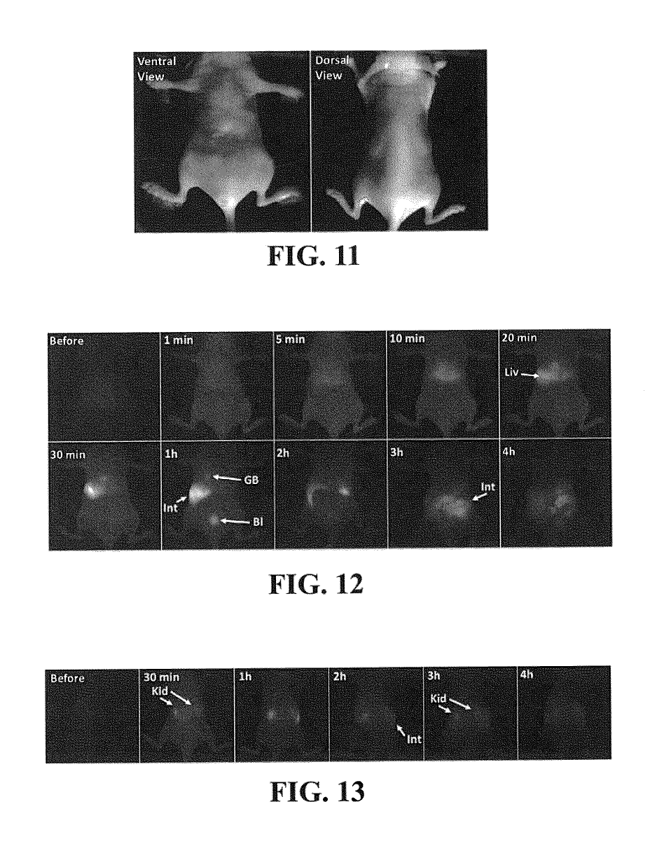

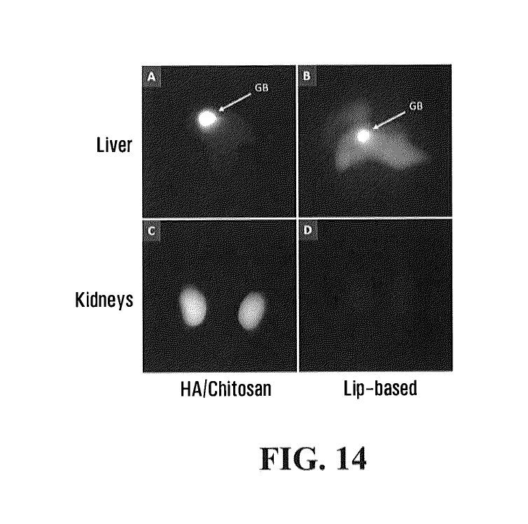

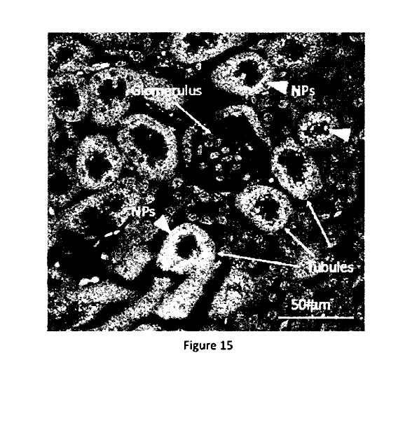

HA-coated NPs with N:P:C=2:1:1.5 were manually prepared using chitosan (DDA=92%, Mn=10 and 40 kDa), labeled siRNA (siRNA-DY677) and HA (Mn=866 kDa) as described in Example 2. Prior to injection of NPs, a volume of trehalose 20% w/v equivalent to the polyplex solution volume was added to reach isotonicity (final concentration of siRNA=33 μg/mL). Invivofectamine NPs were prepared as described in Example 6. Freshly mixed NPs solution was injected in a lateral tail vein of a nude mouse (J:NU 007850). The injected volume was adjusted to the animal weight (10 μL/gram of the animal), in order to obtain a siRNA-DY677 dose of 0.33 and 0.55 mg/kg for CS-based NPs and Invivofectamine NPs, respectively. The DY677 fluorescence was detected by near-infrared fluorescence (NIRF) imaging in the reflection mode. The labeled siRNA was tracked in vivo for 4 hours. Images were acquired in both the ventral and dorsal views (FIG. 11). The animal was anesthetized (inhaled isoflurane) during image acquisitions. On the ventral view, the label was firstly detected in the liver, and then in the intestines, in the gall bladder, and in the bladder (FIG. 12). On the dorsal view (FIG. 13), the label was mainly detected in the kidneys and in the intestines. Thus, the in vivo NIRF images showed evidences of elimination via both the hepatobiliary/intestinal tract (fluorescence in the liver, in the gall bladder, and in the intestines) and the urinary system (fluorescence in the kidneys and in the bladder). After the last in vivo image acquisition (at 4 hours post-injection), the mouse was euthanized by transcardiac perfusion under anesthesia (inhaled isoflurane). The perfusion was performed firstly with 0.9% sodium chloride, then with 10% neutral buffered formalin (NBF). Harvested organs (liver, lungs, kidneys, pancreas, brain, gonads, spleen, heart, and bladder) were individually imaged with the NIRF imager, then preserved in 10% NBF for further analysis. High fluorescence intensity was observed in the gall bladder (FIG. 14A) and in the kidneys (FIG. 14C); low fluorescence was observed in the liver (FIG. 14A), and no fluorescence was detected in other organs (not shown). The intensity in the kidneys was higher when using the 10 kDa chitosan, than when using the 40 kDa (not shown, result to be confirmed with additional animals). Different from the HA-coated NPs, lipid-based NPs (Invivofectamine) showed major accumulation in the liver (FIG. 14B) in addition to the gall bladder, and only a barely detectable accumulation in the kidneys (FIG. 14D).

### Example 9

### Experimental Assay for Studying the In Vivo Delivery of siRNA Using Chitosan and Chitosan Coated Delivery Systems

One approach to study the delivery of siRNA using chitosan and chitosan coated delivery systems would be by preparing the chitosan-based polyplex formulations either manually or with in-line mixing systems. All chitosan-based polyplex formulations would be FD and concentrated upon rehydration then diluted to reach required doses, as described in previous examples. Fluorescent protein (FP) could be either enhanced green fluorescent protein (eGFP) or enhanced cyan fluorescent protein (eCFP).

In order to assess in vivo efficacy of chitosan and chitosan-HA coated systems to deliver siRNA at clinically relevant doses and to promote target specific gene silencing in the kidney, the following proof of concept study (POC) was designed. The POC study is divided into two main studies: a dose finding and an efficacy study per se. Specific gene silencing is assessed by the absence of target silencing when treating the animals with non-targeting siRNA; also known in the art as scrambled siRNA. siRNA may be referred to as non-targeting siRNA or siRNA NT or NT siRNA. Transgenic mice constitutively expressing a fluorescent protein (FP) would be injected with chitosan and chitosan-HA systems formulated with anti-FP siRNA. For the uncoated system, chitosan formulation 92-10 [DDA, Mn] would be complexed to anti-FP siRNA at a N:P ratio of 5. The tested doses that could be considered are 0.5, 1 and 2.5 mg/kg respectively. For the HA-coated system, chitosan formulation 92-10 [DDA, Mn] would be formulated with anti-FP siRNA at an N:P:C ratio of 2:1:1.5. The tested doses would be 1.5 and 7 mg/kg.

A group of 5 mice would be injected per dose per system i.e., uncoated versus coated systems. The injection would be performed by intravenous injection into the tail vein. The mice would be sacrificed 48 hours post injection and the organs viz. lungs, liver, kidney, spleen and heart would be collected and ex-vivo imaged prior to dissection and analysis.

Following ex-vivo imaging, organs would be longitudinally dissected into halves. The first half would be fixed in 4% formaldehyde then paraffin embedded for microscopy whereas the second half of each organ would be stored in RNA Later® and processed for gene expression and northern blotting. Total RNA extraction would be performed on the collected organs. Total RNA would be quantified and analyzed for its integrity before performing Northern blotting. One microgram of the remaining total RNA would be reverse transcribed using the VILO superscript transcription kit using the manufacturer protocol. qPCR would be performed using gene specific primer-probe pairs and data normalized to appropriate reference genes. The delta Cq would be then calibrated to the non-treated control in order to determine the percentage of transgene silencing. Paraffin embedded sections of the organs would be immunohistochemically stained with anti-megalin antibodies and visualized under a confocal microscope following nucleus counter staining. The percentage of positive cells would be scored and compared to the non-treated control. The mean fluorescence intensity of each visual field would be computed and compared to the non-treated control. Histopathology on all organs would be assessed for safety. Blood would be collected via cardiac puncture and serum separated for chemistry and cytokine induction. The type of treatment, mice, formulation tested, doses and number of animals per treatment for the dose-escalation study may be as depicted in Table 9 below.

TABLE 9
Potential group classification for treatments, formulation, dose and number of
animals/treatment for the dose escalation study.

| | | | | # of |
|---|---|---|---|---|
| | | | Dose | animals/ |
| Treatment | Mice | Formulation | (mg/kg) | treatment |
| Non-applicable | C57BL/6 | Non-applicable | 0 | 5 |
| PBS | Example of Transgenic | Non-applicable | 0 | 5 |
| Lipid control | strain that can be used: | Non-applicable | 7 | 5 |
| CS/non-targeting siRNA | C57BL/6-Tg(CAG- | 92-10 | 2.5 | 3 |
| HA/CS/non-targeting siRNA | EGFP)1Osb/J | 92-10 | 7 | 3 |
| CS/anti-FP siRNA | | 92-10 | 0.5 | 5 |
| | | 92-10 | 1 | 5 |
| | | 92-10 | 2.5 | 5 |
| HA/CS/anti-FP siRNA | | 92-10 | 1 | 5 |
| | | 92-10 | 5 | 5 |
| | | 92-10 | 7 | 5 |

| | |
|---|---|
| Total number of animals in the dose finding study | 51 |

Proof of Concept Study to Determine Efficacy of Uncoated and Coated Systems

For the efficacy study, the best performing doses (e.g., but not limited to 2.5 and 7 mg/kg) for uncoated versus coated systems would be selected from Example 10. The doses purported in this example would be selected based on the expected outcomes listed in this Example. Transgenic mice constitutively expressing a fluorescent protein (FP) would be injected with the above said optimal doses. For uncoated systems, chitosan at different molecular weights (Mn 10, 40 and 120 kDa) would be complexed with siRNA at a N:P ratio of 5. For HA-coated systems, chitosan at different molecular weights (Mn 10, 40 and 120 kDa) would be complexed with siRNA at a N:P ratio of 2 and coated with HA for a final N:P:C ratio of 2:1:1.5. Mice would be injected via the tail vain with a single dose. Table 10 shows potential treatments, formulations, doses and number of animals/dose.

TABLE 10
Potential group classification for treatments, formulation, dose and number of
animals/treatment for the efficiency study in reporter transgenic models.

| Group | | | | | # of |
|---|---|---|---|---|---|
| # | Treatment | Mice | Formulation | Dose (mg/kg) | animals |
| 1 | Non-applicable | C57BL/6 | N/A | 0 | 5 |
| 2 | PBS | Example of | N/A | 0 | 5 |
| 3 | Naked siRNA | Transgenic strain | N/A | Optimal dose | 5 |
| 4 | Lipid control | that may be used: | N/A | selected from dose- | 5 |
| 5 | CS/siRNA NT | C57BL/6-Tg(CAG- | 92-120 | escalation | 5 |
| 6 | HA/CS/siRNA NT | EGFP)1Osb/J | 92-120 | In this example | 5 |
| 7 | CS/siRNA anti-FP | | 92-10 | doses are 2.5 and | 5 |
| 8 | CS/siRNA anti-FP | | 92-40 | 7 mg/kg for | 5 |
| 9 | CS siRNA anti-FP | | 92-120 | uncoated versus | 5 |
| 10 | HA/CS/siRNA | | 92-10 | coated systems | 5 |
| | anti-FP | | | | |
| 11 | HA/CS/sRNA | | 92-40 | | 5 |
| | anti-FP | | | | |
| 12 | HA/CS/siRNA | | 92-120 | | 5 |
| | anti-FP | | | | |

| | |
|---|---|
| Number of animals in the efficacy study | 60 |

A group of 5 mice would be injected per dose per system i.e. uncoated versus coated systems. The mice would be sacrificed 48 hours post injection and the organs viz. lungs, liver, kidney, spleen and heart will be collected and ex-vivo imaged prior to dissection and analysis.

### Example 10

### Experimental Protocol for Studying Chitosan and Chitosan-HA Systems in the Treatment of Kidney Diseases Using Target Specific siRNAs

Renal fibrosis is regarded as the final common pathway for most forms of progressive renal disease, and involves glomerular sclerosis and/or interstitial fibrosis. Unilateral ureteral obstruction (UOO) is a well-established experimental model of renal injury leading to interstitial fibrosis. This model could be used as an experimental model that would be reflective of human kidney diseases. UUO is induced by surgically occluding the ureter beneath one of the two kidneys until damage occur. Long term occlusion leads to diseases that mimic chronic kidney pathologies whereas short term occlusion mimics acute kidney diseases. The formulations to be assayed in this protocol would be selected from expected outcomes/results of Example 6.

For the purpose of this experimental protocol, the optimal dose would be expected to be, between about 2.5 and 7 mg/kg (Example 6) for uncoated and HA-coated systems respectively. Two types of injections would be performed, namely intraperitoneal (IP) and tail vein (IV) (Table 11). The two chitosan systems would be formulated with anti-smad3 and/or anti-smad4 siRNA for IV injection and anti-cox2 siRNA for IP injection (Table 11). Five C57BL/6 mice per group would be pre-injected with doses mentioned above at day 3 before surgery. At day of surgery, C57BL/6 mice would be anesthetized using sevofluorane and surgically operated. A midline abdominal incision would be opened and the left ureter exposed and occluded with a 6-0 silk ligature. After recovery, the mice would be injected at day 0, 3 and 5 post surgery and euthanized at day 7. Whole blood would be collected via cardiac puncture and organs viz. heart, lungs, spleen, liver and kidney would be collected for further analysis. Tissue and organ processing, northern blotting and quantitative real time PCR would be performed as in Example.

Additionally to anti-megalin immunostraining described as described herein, paraffin embedded sections of the obstructed and the contralateral kidney from mice injected with anti-smad3/4 siRNA (IV) would be stained with anti-SMAD3, anti-SMAD4, anti-α-SMA, anti-ColIa and anti-ColIIIa antibodies and visualized under a confocal microscope following nucleus counter staining. Immunohistochemistry of renal sections from mice treated with anti-COX2 siRNA (IP) would be stained with anti-COX2, anti-Mac2, anti-ColIa, anti-ColIIIa and anti-α-SMA antibodies.

TABLE 11
Potyential group classification for treatments, formulation, dose and number of animals/treatment
for chitosan delivered siRNA that could be tested for treatment of acute kidney diseases.

| Group | treatment | mice | Formulation | Dose (mg/kg) | | # of animals | Injection |
|---|---|---|---|---|---|---|---|
| 1 | Sham operated | C57BL/6 | N/A | 0 | | 5 | N/A |
| 2 | Naked siRNA | | N/A | 0 | | 5 | IV |
| 3 | CS/siRNA Non | | To be selected from | 2.5 | mg/kg | 5 | IV |
| | targeting | | Example 13. The | | | | |
| 4 | HA/CS/siRNA non- | | formulation viz. Mn | 7 | mg/kg | 5 | IV |
| | targeting | | 120 may be different | | | | |
| 5 | CS/siRNA | | for coated versus | 2.5 | mg/kg | 5 | IV |
| | SMAD3/4 | | uncoated system | | | | |
| 6 | CS/siRNA COX2 | | | 2.5 | mg/kg | 5 | IP |
| 7 | HA/CS/siRNA | | | 7 | mg/kg | 5 | IV |
| | SMAD3/4 | | | | | | |
| 8 | HA/CS/siRNA | | | 7 | mg/kg | 5 | IP |
| | COX2 | | | | | | |

| | |
|---|---|
| Number of animals in the UUO study | 40 |

### Example 11

### Experimental Protocol for Studying Chitosan and Chitosan-HA Systems in Delivering Antagomirs and Potential Use in Treatment of Acute Kidney Diseases

This experiment protocol is designed to assess the use of chitosan and chitosan-HA in the delivery of antagomirs (e.g., anti-miR21) to the kidney. The experiment is based on the UUO model described above. The formulations tested in this protocol would be selected from expected outcomes/results outlined in Example 10. The optimal doses to be tested in this protocol would be expected to be between about 2.5 and about 7 mg/kg (Example 10) for uncoated and HA-coated systems respectively. Five, 10 week old C57BL/6 mice per group would be pre-injected with a dose between about 2.5 mg/kg and about 7 mg/kg of uncoated and HA coated chitosan-anti-miR21 nanoparticle respectively at day 3 before surgery (Table 12). All surgical procedures, injections schedule, euthanasia and blood/organ collection would be conducted as described in Example 7 above. Tissue and organ processing, northern blotting and quantitative real time PCR would be performed as in Example 10. qPCR would be performed against ColIa and ColIIIa genes. In addition to anti-megalin immunostaining, paraffin embedded sections of the obstructed and contralateral kidneys would be immuno stained with anti-ColIa and ColIIIa antibodies and visualized under a confocal microscope following nucleus counter staining.

TABLE 12
Potential group classification for treatments, formulation, dose
and number of animals/treatment for delivery of antagomir study.

| Group # | treatment | mice | Formulation | Dose (mg/kg) | | # of animals | Injection |
|---|---|---|---|---|---|---|---|
| 1 | Sham operated | C57BL/6 | N/A | 0 | | 5 | N/A |
| 2 | Naked anti-miR21 | | N/A | 7 | mg/kg | 5 | IV |
| 3 | CS/non-targeting | | To be selected from | 2.5 | mg/kg | 5 | IV |
| | anti-miR21 | | Example 13. The | | | | |
| 4 | HA/CS/non-targeting | | formulation viz. Mn | 7 | mg/kg | 5 | IV |
| | anti-miR21 | | may be different | | | | |
| 5 | CS/anti-miR21 | | for coated versus | 2.5 | mg/kg | 5 | IV |
| 6 | HA/CS/anti-miR21 | | uncoated system | 7 | mg/kg | 5 | IV |

| | |
|---|---|
| Number of animals in the UUO antagomir study | 30 |

### Example 12

### Coated Delivery Systems Accumulate Predominantly in Proximal Tubule Endothelial Cells in Nude Mice after Intravenous Injection

Preparation and Injection of NPs Solution:

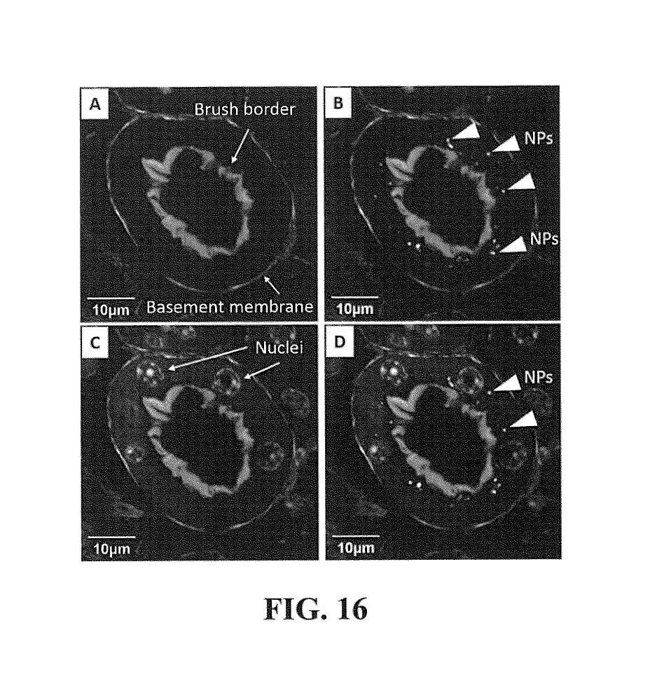

Nanoparticles were prepared using siRNA-DY677 and injected in a lateral tail vein of a nude mouse (J:NU 007850), as described herein. The following formulations were injected: HA/CS92-10/siRNA-DY677 (0.33 mg/kg), HA/CS92-40/siRNA-DY677 (0.33 mg/kg), HA/CS92-120/siRNA-DY677 (0.17 mg/kg), uncoated CS92-10/siRNA-DY677 (0.33 and 0.55 mg/kg), uncoated CS92-40/siRNA-DY677 (0.33 and 0.55 mg/kg), uncoated CS92-120/siRNA-DY677 (0.33 and 0.55 mg/kg). Naked siRNA-DY677 was used as control (0.55 mg/kg). Animals were sacrificed 4 hours after injection and kidneys, where NPs predominantly accumulated, were collected and fixed in 10% NBF. Each kidney was cut transversally in two halves: one half was embedded in sucrose/OCT compound, frozen and cut (10 μm thick), and the other one was embedded in paraffin and cut (6 μm thick). Frozen sections were stained with DAPI (nucleus) and AF488-phalloidin (actin) while paraffin sections were mounted unstained in Permount. Sections were imaged by confocal microscopy. Images show that HA/CS92-10/siRNA-DY677 NPs accumulate predominantly into PTECs where a high siRNA-DY677 fluorescence intensity was observed (arrowheads in FIGS. 15 and 16). Other chitosan-based formulations tested showed similar accumulation profile in PTECs (data not shown). Naked siRNA-DY677 fluorescence signal was either not detectable or very faint in frozen or paraffin sections, respectively (data not shown). Predominant accumulation of chitosan-based NPs in PTECs indicate that they are good candidates for treatment of renal fibrosis. In renal injury, TGF-β is upregulated in the renal tubular epithelium of the nephron including the proximal tubule. The inhibition of TGF-β specifically in the renal tubules prevents damaging interstitial fibrosis. Chitosan-based NPs are good candidates to target TGF-β and any component of its downstream pathway signaling molecules (SMAD effector proteins) specifically in renal tubular epithelial cells (PTECs). The specific inhibition of SMAD3 and SMAD4 prevents the damaging fibrotic response. Other potential targets to inhibit for the treatment of renal fibrosis and transplantation with chitosan-based systems include BAD, BAX, CASP3, MMP9, TNF, MMP8, meosin, MAPK 1 and 14 and. Predominant accumulation of chitosan-based NPs in PTECs indicate that they are good candidates for treatment of renal cell carcinoma (RCC). Most clear cell RCC (ccRCC) arise from a proximal tubular origin as evidenced by positive immunoreactivity to proteins that are normally expressed on PTECs. Potential targets for the treatment of RCC with chitosan-based systems include the delivery of IVT mRNA encoding for pVHL (von Hippel-Lindau) and IVT mRNA encoding for Herpes Simplex Virus-Tymidine Kinase (HSV-TK) suicide gene.

### Example 13

### Chitosan Based Nanoparticles Promote Efficient In Vitro Transfection of IVT Transcribed mRNA

Preparation of IVT mRNA Nucleic Acid Solution:

Fully modified Ψ-uridine and 5-methylcytidine in vitro transcribed mRNA encoding the firefly luciferase (FLuc mRNA 5meC, Ψ, cat# L-6107) was bought from TriLink Biotechnologies Inc. The FLuc mRNA was obtained as a solution in 10 mM Tris-HCL, pH 7.5 at a concentration of 1 mg/mL and refered herein as FLuc stock solution. The FLuc stock solution was diluted at a final using sterile RNase free water to obtain a final concentration of 0.1 mg/mL.

Preparation of Chitosan Solution:

Fully characterized chitosan with specific DDA and MW (Table 13) were dissolved in 1N HCl overnight at room temperature to obtain a final chitosan concentration of 5 mg/mL and filtered using 0.2 μm filter.

TABLE 13
Chitosan formulations

| CS formulations | CS (mg) | H₂O (μL) | HCl 1N (μL) |
|---|---|---|---|
| 80-10 | 3.40 | 664 | 16 |
| 98-5 | 3.65 | 707 | 22 |
| 98-10 | 2.29 | 443 | 13.8 |
| 92-5 | 4.84 | 941 | 27 |
| 92-10 | 2.48 | 481 | 13.8 |
| 92-120 | 6.04 | 1174 | 33.8 |

Manual Production of Polyplexes: Uncoated Chitosans/FLuc mRNA Samples:

Chitosan-Fluc mRNA complexes were prepared at an N:P ratio of 5:1, (N:P is the molar ratio of amines (N) of chitosan, to the phosphates (P) of the nucleic acid) according to the table below (Table 14):

TABLE 14
Composition of polyplexes

| | | | CS stock | | | |
|---|---|---|---|---|---|---|
| CS | N:P | Total Volume | solution | H₂O | [N] | [P] |
| formulations | ratio | (mL) | (μL) | (μL) | (mM) | (mM) |
| 80-10 | 5 | 1 | 66 | 934 | 1.56 | 0.312 |
| 98-5 | 5 | 1 | 52 | 948 | 1.56 | 0.312 |
| 98-10 | 5 | 1 | 52 | 948 | 1.56 | 0.312 |
| 92-5 | 5 | 1 | 56 | 944 | 1.56 | 0.312 |
| 92-10 | 5 | 1 | 56 | 944 | 1.56 | 0.312 |
| 92-120 | 5 | 1 | 56 | 944 | 1.56 | 0.312 |

For each formulation, a volume of 20 μL of FLuc mRNA working solution (0.1 mg/mL) was pipetted and transferred to a 0.6 mL RNase free centrifuge tube. A volume of 20 μL of the chitosan solution (N:P 5) was pipetted into the FLuc mRNA solution and the mixture was immediately pipetted up and down ˜10× for homogenization. Final volume of the prepared solution 40 μL. The prepared polyplex solution was incubated for 30 minutes at room temperature before transfection. For transfection, the HEK293 cell line was seeded in a 96 well plate at a density of 40 000 cell/well 24 hour prior to transfection. On the day of transfection, cell confluence reached around 80%. For transfection, media over cells was removed and replaced with 98 μl of transfection media (DMEM-HG, pH 6.5, no serum). For transfection, a volume of 2 μL (equivalent to 100 ng of IVT mRNA) of chitosan IVT FLuc mRNA polyplexes was added into each well and the plate incubated for 4 hours. Media over cells was aspirated and wells replenished with complete medium and incubated for an extra 44 hours before analysis. Dynamic Light Scattering (DLS) was used to measure the size and polydispersity of the prepared nanoparticles (polyplexes). The remaining volume of polyplexes prepared for transfection was diluted 1:1 in water for a final volume of 76 μL and subjected for DLS measurement. The instrument was adjusted for three size measurements on each sample. Mean values of Z-Average, mean intensity-weighted size and PdI (average of the three DLS readings performed for each sample) were calculated. The standard deviation and also the coefficient of variation (CV %) were calculated for all samples. Transmission Electron Microscopy (TEM) was used to assess size and morphology of the polyplexes. For TEM analyses, a drop of solution was pipetted on the TEM grid then the excess sample solution was dried by capillarity on a filter paper. For the assessment of nanoparticle stability, chitosan 92-10 was used to prepare nanoparticles with siRNA and mRNA at different N:P ratio. The N:P ratios were prepared as per Table 15 below.

TABLE 15
Composition of nanoparticles

| Chitosan | N:P | Vol. chitosan (μL) | Vol. H₂O (μL) |
|---|---|---|---|
| 92-10 | 0.5 | 6 | 994 |
| | 2 | 22 | 978 |
| | 5 | 56 | 944 |
| | 10 | 112 | 888 |

For the competition assay, heparin stock solution was prepared at 1 mg·mL by dissolving 1 mg of heparin sodium salt in 1 mL of nuclease-free water; filter sterilized through a 0.2 μm filter. The heparin working solution was prepared in 25 mM MES pH 6.5 as indicated in Table 16:

TABLE 16
Composition of heparin working solution

| Heparin working | 25 mM MES pH 6.5 | Heparin Stock solution |
|---|---|---|
| Concentration | (μL) | (μL) |
| 7.75 μg/mL | 2980 | 22.2 |
| 18.57 μg/mL | 2947 | 55.6 |

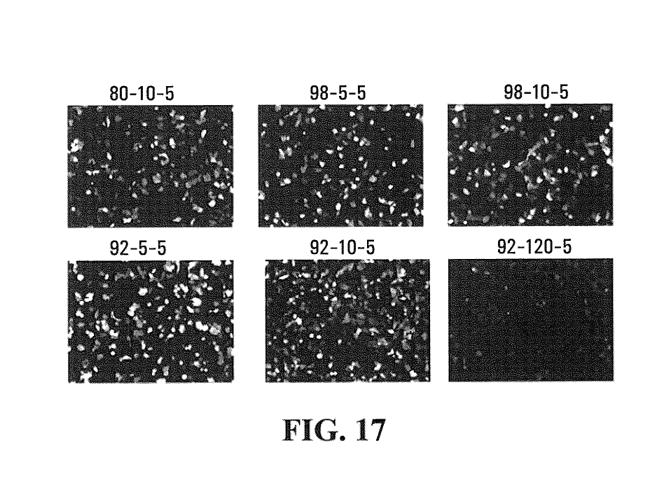

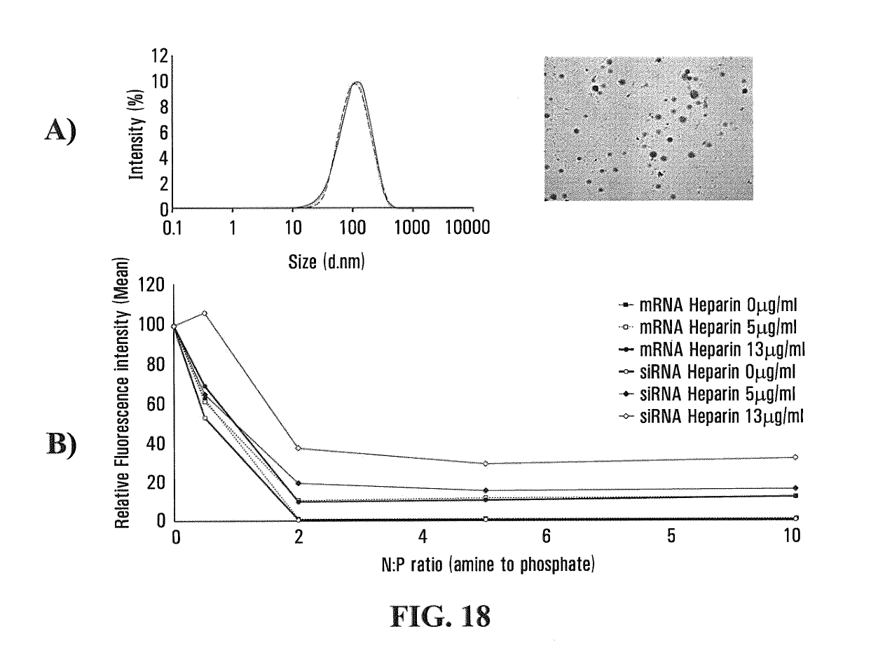

The chitosan siRNA and chitosan mRNA nanoparticles were prepared as described above. Briefly, the 30 μL of nanoparticles were prepared for both siRNA and mRNA by manually mixing chitosan to nucleic acid at a 1:1 ratio. Following complexation, the nanoparticle were further diluted in 25 mM MES (pH 6.5), incubated with heparin for 60 minutes and further diluted at 1:1 ratio with RiboGreen. The RiboGreen reagent was prepared as per the manufacturer protocol. Fluorescence measurements were taken 5 minutes following RiboGreen addition using a TECAN Infinite M500 system. Following transfection, cells were imaged using an epi-fluorescence microscope. Images were taken both under fluorescence excitation and DIC. The data reveal efficient transfection for low molecular weight chitosans for all tested degrees of deacetylation (FIG. 17). The comparison of low versus high MW chitosan (DDA 92%, MW 10 versus 120) show that high molecular weights are inefficient at transfecting cells (FIG. 17). This is reminiscent to previous findings where high molecular weight chitosan is inefficient at transfecting plasmid DNA and contrary to our finding with siRNA where high molecular weight chitosan were efficient at delivering siRNA both in vitro and in vivo. Complexes were analyzed in DLS, after dilution in water. Manually mixed chitosan-mRNA nanoparticles as size range between 80-100 nm depending on the composition. FIG. 18A show nanoparticle prepared with a chitosan 92-10 at an N:P ratio of 5. The average nanoparticle size is around 90 nm. TEM imaging of the nanoparticles demonstrate spherical shaped nanoparticles FIG. 18A that are reminiscent to our chitosan-siRNA nanoparticles. In the absence of heparin, full complexation efficiency was achieved for both mRNA siRNA at an N:P ratio of 2. As expected, increasing the N:P ratio has no influence on the complexation efficiency. The addition of 13 μg/mL of heparin strongly destabilized chitosan siRNA nanoparticles by lowering the complexation efficiency from 100 to 60% (FIG. 18B). A slight improvement of complexation efficiency (˜10%) was observed when N:P ratio was increased from 2 to 5 followed by a plateau. For mRNA, the addition of heparin (13 μg/mL) induced a less marked destabilization of the nanoparticle with a loss of efficiency around 20%; two fold less that what was observed with siRNA (FIG. 18B). Taken together, these results show that chitosan-mRNA are more stable than chitosan-siRNA in presence of competing polyanions. This is probably due to the physico-chemical properties of the two nucleic acids used i.e. length, structure, etc.

### Example 14

### Experimental Protocol for Studying Delivery of Antagomirs to Specific Target Using the Chitosan-Coated Delivery Systems

The delivery systems of the present disclosure could be tested for their capacity in delivering antagomirs to specific targets/organs. As such, the delivery systems of the present disclosure could be useful in the study and potentially in the prevention and/or treatment of rare genetic kidney diseases. One way such could be performed would be by testing the delivery systems of the present disclosure in the Alport mouse model. The Alport mouse model mimics the human disease and exhibit a time-dependent increase in Albumin to Creatinine ratio (ACR) and Blood urea nitrogen ratio (BUN) reaching around 40 mg/mg and 125 mg/dl respectively at week 15. The GFR is reduced by 80% at week 15. The formulations of the present disclosure to be tested in such study would be selected from expected outcomes/results listed in Example 6. For the purpose of this example, the expected optimal dose for HA coated systems would be compared to the dose of about 25 mg/kg of naked anti-miR21 (Table 17). The naked miR21 dose of 25 mg/kg (group 3, Table 17) would be selected from the art since it shows clinical relevance in the Alport model. Five four week old F1 hybrid (B6, 129) Col4a3−/− mice would be injected twice a week with HA-coated chitosan-antimiR21 nanoparticles at a dose of about 7 mg/kg via tail vain injection. The schedule of injection would be conducted until week 15. Mice would then be euthanized and organs would be collected for histological analysis. Blood would be drawn at weeks 6, 9, 12 and 15 to monitor blood urea ratio and creatinine to albumin ratio. Another group of F1 hybrid (B6, 129) Col4a3−/− mice would be injected as above mentioned, left for normal death in order to assess the improvement of survival free disease. Ramipril is an angiotensin converting enzyme inhibitor (ACE) used for the treatment of high blood pressure and diabetes associated kidney diseases. At low doses, it is used for the prevention of kidney damage.

TABLE 17
Potential group classification for treatments, formulation, dose and
number of animals/treatment for studying rare genetic disorders.

| Group # | treatment | mice | Formulation | Dose (mg/kg) | | # of animals | Injection |
|---|---|---|---|---|---|---|---|
| 1 | Non-treated | Wild type | | | | 5 | N/A |
| 2 | Non-treated | F1 hybrid | N/A | 0 | | 5 | N/A |
| 3 | Anti-miR21 naked | (B6, 129) | N/A | 25 | mg/kg | 5 | IV |
| 4 | HA/CS/scrambled | Col4a3 | To be selected | 7 | mg/kg | 5 | IV |
| | anti-miR21 | | Example 13. The | | | | |
| 5 | HA/CS/anti-miR21 | | formulation viz. Mn | 7 | mg/kg | 5 | IV |
| (survival free | | | may be different for | | | | |
| assessment) | | | coated versus | | | | |
| | | | uncoated system | | | | |
| 6 | HA/CS/anti-miR21 + | | N/A | 7 | mg/kg | 5 | IV |
| | Ramipril | | | | | | |

| | |
|---|---|
| Number of animals in the Alport antagomir study | 30 |

### Example 15

### Preparation of HA-Coated Chitosan/ODN Complexes with an Automated in-Line Mixing System—Large-Scale Production

Preparation of Nucleic Acid Solution

A 200 μg/ml ODN (ApoB) solution with trehalose and L-histidine concentrations of 0.5% w/v and 3.5 mM, respectively, was prepared as described in Example 2.

Preparation of Chitosan Solution

A 225 μg/ml chitosan (Mn 10 kDa, 92% DDA) solution with trehalose and L-histidine concentrations of 0.5% w/v and 3.5 mM, respectively, was prepared as described in Example 2.

Preparation of HA Solution

A 390 μg/mL HA (Mn=866 kDa) solution with trehalose and L-histidine concentrations of 0.5% w/v and 3.5 mM, respectively, was prepared as described in Example 2.

Automated Production of Complexes

Mixing platform design: The mixing platform is established as described in Example 3.

Washing: The vessels, connections, and tubings of the in-line mixing system were cleaned/washed as described in Example 3.

Priming: The system was primed following the procedure described in Example 3.

Discontinuous mixing: Discontinuous mixing (MODE 2 of Example 3) was used for large scale production of HA-coated NPs. Each pump was controlled to dispense and mix 10 mL of ODN solution with 10 mL of CS solution with flow rates of 150 mL/min for each solution. The produced uncoated complex solution was kept for 30 minutes at room temperature, then, using the same in-line mixing configuration, 8 mL of HA solution was mixed with 16 mL of the CS/ODN complex solution using flow rates of 75 and 150 mL/min, respectively. The complexes were produced with N:P:C=2:1.5:1.

Manual Preparation of Complexes

The manually mixed sample was prepared as described in Example 2. The complexes were produced with N:P:C=2:1.5:1.

DLS Measurements

Samples were analyzed for their size, PDI and zeta potential as described in Example 3.

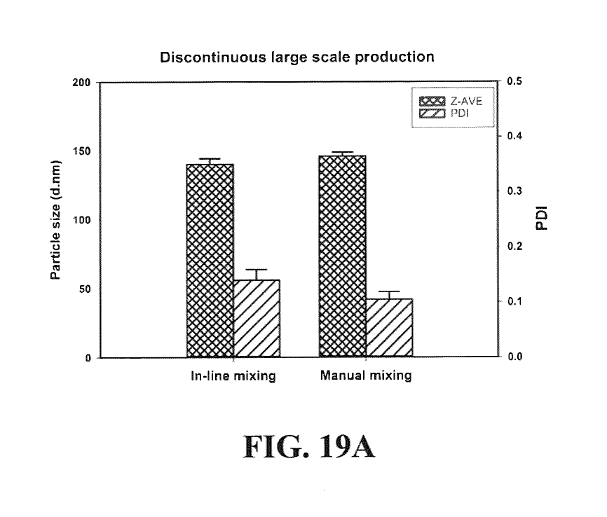

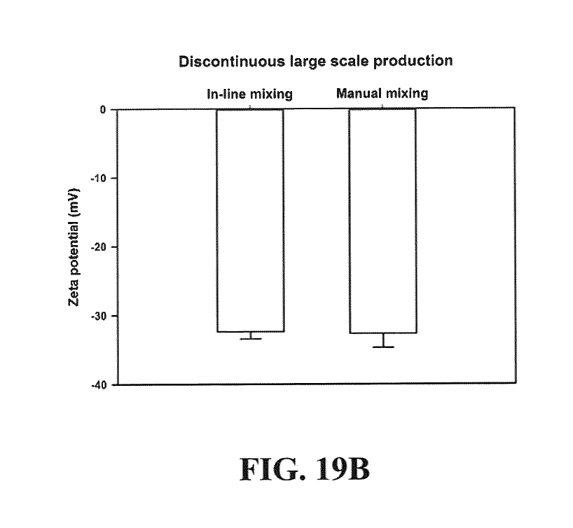

Complexes produced in-line through discontinuous mixing mode were not aggregated and had size comparable to that of manually prepared particles (FIGS. 19A-19B).

Particles produced using discontinuous in-line mixing method were small-sized and homogeneous with low PDI of 0.14 (FIGS. 19A-19B). The preparation of complexes using the same mixing volumes but with continuous mixing (MODE 1) resulted in macroscopic aggregation (data not shown).

### Example 16

### HA-Coated ODN/CS Complexes May be Prepared and Freeze-Dried at Neutral pH

Preparation of Nucleic Acid Solution

A 200 μg/ml ODN (ApoB) solution with trehalose concentration of 0.5% w/v, was prepared as described in Example 2, but without addition of L-histidine.

Preparation of Chitosan Solution

A 225 μg/ml chitosan (Mₙ 10 kDa, 92% DDA) solution with trehalose concentration of 0.5% w/v, was prepared as described in Example 2, but without addition of L-histidine.

Preparation of HA Solution

Sodium hyaluronate with molecular weight (Mn) of 866 kDa was dissolved in RNase free water at room temperature at 1.25 mg/mL. The HA stock solution was diluted to 390 μg/mL using RNase free water, filtered 4% (w/v) trehalose dihydrate, and filtered 28 mM L-histidine buffer at pH 7.5, and sterilized using 0.2 μm filter. Trehalose and L-histidine final concentrations were 0.5% w/v and 6 mM, respectively.

Preparation of HA Coated Chitosan/siRNA Complexes

Fresh sample: 100 μL of chitosan solution, 100 μL of ODN solution, and 100 μL of HA solution were mixed manually as described in Example 2. Preparation of complexes was done twice. Samples to be freeze-dried and rehydrated to 1× the initial concentration: 160 μL of chitosan solution, 160 μL of ODN solution, and 160 μL of HA solution were mixed manually as described in Example 2. Preparation of complexes was done twice. Samples to be freeze-dried and rehydrated to 20× the initial concentration: 160 μL of chitosan solution, 160 μL of ODN solution, and 160 μL of HA solution were mixed manually as described in Example 2. Preparation of complexes was done twice. pH of all complexes prepared as described above was near neutral (˜7±0.1)

Sample Freeze-Drying

Samples were freeze-dried in 2 mL glass serum vials (400 μL per vial), as described in Example 4.

DLS Measurements

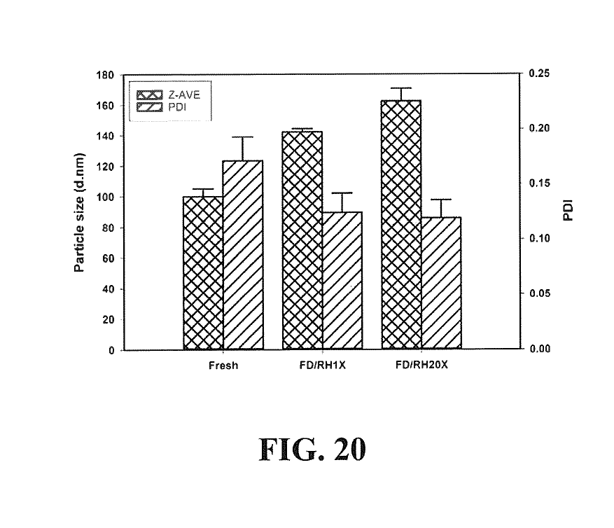

Six samples were analyzed for their size and PDI upon preparation: two freshly prepared and four following a freeze-drying and rehydration to either initial volume or reduced volumes of 20 μL of RNase free water for concentration factor of 20×. Upon rehydration, each sample was left untouched for 10 to 15 minutes to stabilize. Then, 20× rehydrated samples were supplemented with RNase free water to their original volume (400 μL) prior to size analysis. Each sample was diluted 8× for DLS analysis: 50 μL of each sample was diluted by adding 350 μL of RNase free water. For each sample, three consecutive size analyses were done. The instrument was operated in automatic mode. Average values of size PDI for different samples were calculated. Nanoparticles were found to be stable at neutral pH and could be freeze-dried to reach the final concentration factor of 1× or 20× without seeing particle aggregation (FIG. 20). Uncoated particles prepared in similar conditions (i.e. neutral pH were found to be significantly aggregated, data not shown)

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

It will be understood by those of skill in the art that throughout the present disclosure, the term “a” used before a term encompasses embodiments containing one or more to what the term refers. It will also be understood by those of skill in the art that throughout the present disclosure, the term “comprising”, which is synonymous with “including”, “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although the present invention has been described in considerable detail with reference to certain embodiments thereof, variations and refinements are possible and will become apparent to persons skilled in the art in light of the present description.

All references cited throughout the specification are hereby incorporated by reference in their entirety for all purposes.

### VII. REFERENCES

1. Al-Qadi, S., M. Alatorre-Meda, E. M. Zaghloul, P. Taboada and C. Remunan-Lopez (2013). “Chitosan-hyaluronic acid nanoparticles for gene silencing: The role of hyaluronic acid on the nanoparticles' formation and activity.” Colloids and Surfaces B-Biointerfaces 103: 615-623.
2. Almalik, A., R. Donno, C. J. Cadman, F. Cellesi, P. J. Day and N. Tirelli (2013). “Hyaluronic acid-coated chitosan nanoparticles: Molecular weight-dependent effects on morphology and hyaluronic acid presentation.” Journal of Controlled Release 172(3): 1142-1150.
3. Boeckle, S., K. von Gersdorff, S. van der Piepen, C. Culmsee, E. Wagner and M. Ogris (2004). “Purification of polyethylenimine polyplexes highlights the role of free polycations in gene transfer.” Journal of Gene Medicine 6(10): 1102-1111.
4. Contreras-Ruiz, L., M. de la Fuente, J. E. Parraga, A. Lopez-Garcia, I. Fernandez, B. Seijo, A. Sanchez, M. Calonge and Y. Diebold (2011). “Intracellular trafficking of hyaluronic acid-chitosan oligomer-based nanoparticles in cultured human ocular surface cells.” Molecular Vision 17(34-35): 279-290.
5. de la Fuente, M., B. Seijo and M. J. Alonso (2008). “Novel hyaluronic acid-chitosan nanoparticles for ocular gene therapy.” Investigative Ophthalmology & Visual Science 49(5): 2016-2024.
6. Duceppe, N. and M. Tabrizian (2009). “Factors influencing the transfection efficiency of ultra low molecular weight chitosan/hyaluronic acid nanoparticles.” Biomaterials 30(13): 2625-2631.
7. Fahrmeir, J., M. Gunther, N. Tietze, E. Wagner and M. Ogris (2007). “Electrophoretic purification of tumor-targeted polyethylenimine-based polyplexes reduces toxic side effects in vivo.” Journal of Controlled Release 122(3): 236-245.
8. Gwak, S.-J., J. K. Jung, S. S. An, H. J. Kim, J. S. Oh, W. A. Pennant, H. Y. Lee, M. H. Kong, K. N. Kim, D. H. Yoon and Y. Ha (2012). “Chitosan/TPP-Hyaluronic Acid Nanoparticles: A New Vehicle for Gene Delivery to the Spinal Cord.” Journal of Biomaterials Science-Polymer Edition 23(11): 1437-1450.
9. Howard, K. A., S. R. Paludan, M. A. Behlke, F. Besenbacher, B. Deleuran and J. Kjems (2009). “Chitosan/siRNA Nanoparticle-mediated TNF-α Knockdown in Peritoneal Macrophages for Anti-inflammatory Treatment in a Murine Arthritis Model.” Molecular Therapy 17(1): 162-168.
10. Howard, K. A., U. L. Rahbek, X. Liu, C. K. Damgaard, S. Z. Glud, M. O. Andersen, M. B. Hovgaard, A. Schmitz, J. R. Nyengaard, F. Besenbacher and J. Kjems (2006). “RNA interference in vitro and in vivo using a novel chitosan/siRNA nanoparticle system.” Mol Ther 14(4): 476-484.
11. Jean, M., M. Alameh, M. D. Buschmann and A. Merzouki (2011). “Effective and safe gene-based delivery of GLP-1 using chitosan/plasmid-DNA therapeutic nanocomplexes in an animal model of type 2 diabetes.” Gene Therapy 18(8): 807-816.
12. Jokerst, J. V., T. Lobovkina, R. N. Zare and S. S. Gambhir (2011). “Nanoparticle PEGylation for imaging and therapy.” Nanomedicine 6(4): 715-728.
13. Judge, A. D., G. Bola, A. C. H. Lee and I. MacLachlan (2006). “Design of noninflammatory synthetic siRNA mediating potent gene silencing in vivo.” Molecular Therapy 13(3): 494-505.
14. Koping-Hoggard, M., K. M. Varum, M. Issa, S. Danielsen, B. E. Christensen, B. T. Stokke and P. Artursson (2004). “Improved chitosan-mediated gene delivery based on easily dissociated chitosan polyplexes of highly defined chitosan oligomers.” Gene Therapy 11(19): 1441-1452.
15. Lavertu, M., S. Methot, N. Tran-Khanh and M. Buschmann (2006). “High efficiency gene transfer using chitosan/DNA nanoparticles with specific combinations of molecular weight and degree of deacetylation.” Bioinaterials 27(27): 4815-4824.
16. Lu, H., L. Lv, Y. Dai, G. Wu, H. Zhao and F. Zhang (2013). “Porous Chitosan Scaffolds with Embedded Hyaluronic Acid/Chitosan/Plasmid-DNA Nanoparticles Encoding TGF-beta 1 Induce DNA Controlled Release, Transfected Chondrocytes, and Promoted Cell Proliferation.” Plos One 8(7).
17. Lu, H. D., H. Q. Zhao, K. Wang and L. L. Lv (2011). “Novel hyaluronic acid-chitosan nanoparticles as non-viral gene delivery vectors targeting osteoarthritis.” International Journal of Pharmaceutics 420(2): 358-365.
18. Ma, P. L., M. D. Buschmann and F. M. Winnik (2010). “Complete Physicochemical Characterization of DNA/Chitosan Complexes by Multiple Detection Using Asymmetrical Flow Field-Flow Fractionation.” Analytical Chemistry 82(23): 9636-9643.
19. Oh, E. J., K. Park, K. S. Kim, J. Kim, J.-A. Yang, J.-H. Kong, M. Y. Lee, A. S. Hoffman and S. K. Hahn (2010). “Target specific and long-acting delivery of protein, peptide, and nucleotide therapeutics using hyaluronic acid derivatives.” Journal of Controlled Release 141(1): 2-12.
20. Oliveira, A. V., D. B. Bitoque and G. A. Silva (2014). “Combining Hyaluronic Acid with Chitosan Enhances Gene Delivery.” Journal of Nanomaterials.
21. Rinaudo, M. (2006). “Chitin and chitosan: Properties and applications.” Progress in Polymer Science 31(7): 603-632.
22. Urban-Klein, B., S. Werth, S. Abuharbeid, F. Czubayko and A. Aigner (2005). “RNAi-mediated gene-targeting through systemic application of polyethylenimine (PEI)-complexed siRNA in vivo.” Gene Therapy 12(5): 461-466.
23. Xu, L. and T. Anchordoquy (2011). “Drug Delivery Trends in Clinical Trials and Translational Medicine: Challenges and Opportunities in the Delivery of Nucleic Acid-Based Therapeutics.” Journal of Pharmaceutical Sciences 100(1): 38-52.
24. Zwart, A., O. W. vanAssendelft, B. S. Bull, S. M. Lewis and W. G. Zijlstra (1996). “Recommendations for reference method for haemoglobinometry in human blood (ICSH standard 1995) and specifications for international haemiglobincyanide standard (4th edition).” Journal of Clinical Patholooy 49(4): 271-274.

1. A coated chitosan-based polyplex, comprising:
i) a chitosan;
ii) a nucleic acid; and
iii) a coating including an additional polyelectrolyte;
the coated chitosan-based polyplex having an initial or a final molar ratio of amine groups of chitosan (N) to phosphate groups of the nucleic acid (P) to carboxyl groups of the additional polyelectrolyte (C) (N:P:C), wherein the N has a value between about 1.0 and about 10.5, the P has a value between about 1.0 and about 2.0 and the C has a value between about 1.0 and 10.5.

2. The coated chitosan-based polyplex of claim 1, wherein the coated chitosan-based polyplex is a coated chitosan-based nanoparticle.

3. The coated chitosan-based polyplex of claim 1, wherein the additional polyelectrolyte is a polyanion.

4. The coated chitosan-based polyplex of claim 3, wherein the polyanion is hyaluronic acid (HA).

5. The coated chitosan-based polyplex of claim 4, wherein the HA has a molecular weight between 2 kDa and 1.5 MDa.

6. The coated chitosan-based polyplex of claim 1, wherein the chitosan has a molecular weight between 2 kDa and 200 kDa.

7. The coated chitosan-based polyplex of claim 1, wherein the chitosan has a deacetylation degree (DDA) between 70% and 100%.

8. The coated chitosan-based polyplex of claim 1, wherein the composition is in freeze-dried form.

9. The coated chitosan-based polyplex of claim 1, wherein the nucleic acid is a deoxyribonucleic acid.

10. The coated chitosan-based polyplex of claim 1, wherein the nucleic acid is a ribonucleic acid.

11. The coated chitosan-based polyplex of claim 9, wherein the nucleic acid is selected from a plasmid DNA, a vector DNA, and a minicircle DNA.

12. The coated chitosan-based polyplex of claim 10, wherein the nucleic acid is selected from an mRNA, an siRNA, and a microRNA.

13. The coated chitosan-based polyplex of claim 10, wherein the nucleic acid is an anti-microRNA.

14. The coated chitosan-based polyplex of claim 13, wherein the anti-microRNA is an antagomir.

15. (canceled)

16. (canceled)

17. (canceled)

18. (canceled)

19. (canceled)

20. A method for delivering a nucleic acid to a target in a subject, the method comprising the steps of administering the coated chitosan-based polyplex of claim 1 to the subject.

21. The method of claim 20, wherein the target is a kidney, a liver, a spleen, a heart, a lymph node, an eye, an ear, a lung, an articulation, or a bladder.

22. The method of claim 20, wherein the target is a kidney.

23. The method of claim 20, wherein the target is a liver.

24. The method of claim 20, wherein the subject suffers from a cancer, renal disease, liver disease, cardiovascular disease, genetic disease, viral disease, neuromuscular disease, neurodegenerative disease, inflammatory disease, Arthritis, metabolic disease or diabetes.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A process for manufacturing the coated chitosan-based polyplex of claim 1, the process comprising
mixing a nucleic acid with a chitosan to obtain a first solution,
mixing the first solution with an additional polyelectrolyte to obtain the coated chitosan-based polyplex.
